(19) United States
(12) Patent Application Publication
(10) Pub. No.: US 2023/0114627 A1
(43) Pub. Date: Apr. 13, 2023

(54) COMPOSITION COMPRISING A METAL ASCORBO-CAMPHORATE COMPOUND AND METHOD FOR PREVENTION AND/OR TREATMENT OF PAPILLOMAVIRUS INFECTIONS

(71) Applicant: MGB PHARMA Inc., Wilmington, DE (US)

(72) Inventors: Bernard Lucien Gombert, Nimes (FR); Aurele Henri Mannarini, Conca (FR)

(21) Appl. No.: 17/991,581

(22) Filed: Nov. 21, 2022

Related U.S. Application Data
(63) Continuation-in-part of application No. PCT/FR2021/050897, filed on May 20, 2021.

(30) Foreign Application Priority Data
May 20, 2020 (FR) FR2005326

(51) Int. Cl. A61K 31/375; A61K 31/315; A61K 9/20; A61K 9/48; A61K 9/00; A61K 31/341

(52) U.S. Cl. CPC A61K 31/375; A61K 31/315; A61K 9/20; A61K 9/4808; A61K 9/0036; A61K 31/341

(57) ABSTRACT

The present invention provides a method of preventive and/or curative treatment of viral infections caused by viruses of the family Papillomaviridae in a subject by administering a therapeutically effective amount of a composition to the subject, wherein the composition comprises of at least one metal ascorbo-camphorate compound, a physiologically acceptable aqueous solvent, and at least one physiologically acceptable excipient, and wherein the metal ascorbo-camphorate compound is Zinc tetra-ascorbo-camphorate referred to as C14. Further, the present invention provides a pharmaceutical composition directed against viral infections caused by viruses of the family Papillomaviridae, the pharmaceutical composition comprising: (a) one metal ascorbo-camphorate compound; (b) a physiologically acceptable aqueous solvent; and (c) at least one physiologically acceptable excipient, wherein the metal ascorbo-camphorate compound is Zinc tetra-ascorbo-camphorate referred to as C14 as well as its use in the preventive and/or curative treatment of viral infections caused by viruses of the family Papillomaviridae. Also provided are a method for the preparation of the said pharmaceutical composition.

### CROSS REFERENCE TO RELATED APPLICATIONS

The current application claims benefit of the PCT Patent Application PCT/FR2021/050897 filed May 20, 2021.

### FIELD OF THE INVENTION

The present invention generally relates to a medicament and method for preventive and/or curative treatment of an infection caused by at least one virus of the family Papillomaviridae, in particular human papillomaviruses (HPV). More specifically, the present invention relates to a medicament or composition and method of prevention or curative treatment of viral infections comprising administration of said medicament or composition targeting human papillomaviruses (HPV), particularly the known high-risk and oncogenic HPVs such as the HPV-16 or HPV-18 genotypes which are the main papillomaviruses involved in the development of malignant tumors.

### BACKGROUND OF THE INVENTION

Human papillomaviruses (HPV) are viruses that belong to the Papillomaviridae family. More than a hundred have been identified to date. A distinction is made between so-called low-risk HPVs, which cause external condylomas, and so-called high-risk HPVs (in particular HPV-16 or HPV-18), which are involved in precancerous lesions and mucocutaneous cancers, particularly of the cervix or, more generally, of the anogenital region.

Papillomaviruses are extensively studied and described in the scientific literature. They are all epitheliotropic viruses that are clinically responsible for benign or malignant lesions or tumors. Structurally, papillomaviruses are small, non-enveloped viruses with a capsid consisting of 72 capsomers including the major L1 protein and the L2 protein.

HPV infects the stem cells of the multi-layered epithelium of mucocutaneous keratinocytes (such as the basal lamina of the ectocervix). The complete HPV multiplication cycle consists of several phases that take place during epithelial differentiation and shedding. After infection of the cells with the virus, the viral DNA remains in latent form for several years in the episomal (circular) form in the nucleus of the host cell. Endogenous and exogenous cofactors activate the viral DNA, which splits and integrates into the host cell genome. Sequential expression of the viral genes then occurs, allowing replication of the viral DNA and subsequent production of new infectious virions. This event leads to the overexpression of the two viral octoproteins, E6 and E7, whose combined action is necessary for the immortalization and transformation of the cell. The cancerous cells lead to dysplasia, which in the worst case can develop into cancer.

Papillomaviruses have a genome consisting of double-stranded, circular DNA of about 8,000 base pairs. HPV DNA contains two open reading frames (OPFs) encoding the L1 and L2 capsomere proteins. When the L1 POL is expressed in vitro, alone or in association with L2, the corresponding proteins self-assemble into viral pseudo particles, commonly referred to as VLPs (Virus Like Particles), also known as pseudovirions or pseudo viruses (PsV), whose morphology and antigenic properties are similar to those of native virions. Viral pseudo particles (VLPs) are the basis for the present prophylactic HPV vaccines.

The international patent application, WO1999/015630A1 relates to papillomavirus-derived pseudo viruses for use in gene therapy or gene immunization. Prophylactic vaccines developed from VLP provide excellent protection against lesions induced by both HPV-16 and HPV-18 involved in 70% of cervical cancers. However, the manufacture of these vaccines is quite complex and remains a challenge in the art.

Currently, the means of treating HPV viral infection are based on surgical treatment of the infected body part following a diagnosis of cancer risk or invasive cancer, or aftermath of prophylactic vaccines. Therefore, there are no drugs other than vaccines available on the market for preventive and/or curative treatment of HPV infections.

Hence, there is still a need for medicines that can be used in the preventive and/or curative treatment of infections by a virus of the papillomavirus family, in particular HPV, and especially those that are oncogenic, also known as high-risk (especially HPV-16 or HPV-18). The present invention is intended to meet this need and provide a solution to the above highlighted problem.

### SUMMARY OF THE INVENTION

The present disclosure provides a method for preventive and/or curative treatment of viral infections caused by viruses of the family Papillomaviridae in a subject, said method comprising the step of administering a therapeutically effective amount of a composition to the subject, wherein the composition comprises at least one metal ascorbo-camphorate compound, a physiologically acceptable aqueous solvent, and at least one physiologically acceptable excipient, and wherein the metal ascorbo-camphorate compound is Zinc tetra-ascorbo-camphorate referred to as C14.

In an aspect of the present invention, a pharmaceutical composition directed against viral infections caused by viruses of the family Papillomaviridae, the pharmaceutical composition comprising: (a) a metal ascorbo-camphorate compound; (b) a physiologically acceptable aqueous solvent; and (c) at least one physiologically acceptable excipient, wherein the metal ascorbo-camphorate compound is a metal tetra-ascorbo-camphorate, wherein the metal ascorbo-camphorate compound comprises a metal ion selected from the group comprising metals of the series 2 of the Mendeleev classification including Zinc, Copper, Gold, Silver, and Magnesium, wherein the metal ascorbo-camphorate compound is soluble in the physiologically acceptable aqueous solvent, wherein the viruses of the family Papillomaviridae are human papilloma viruses (HPV) including oncogenic papillomaviruses consisting of the HPV-16 genotype, the HPV-18 genotype, or a combination thereof, and wherein the at least one physiologically acceptable excipient comprised in said composition provides the packaging for administration via a route of administration in a subject for a route of administration selected from a group consisting of intravenous route, intraperitoneal route, oral route, intranasal route, subcutaneous route, intrathecal route, intraventricular route, intramuscular route, by instillation route via a vaginal bulb and/or pump, by perfusion route, and by vaginal ring route.

In an aspect of the present invention, a method for preparation of a pharmaceutical composition for administering a therapeutically effective amount of said composition to a subject directed against viral infections caused by viruses of the family Papillomaviridae, said method comprising the steps of: preparing camphoric acid by oxidation of camphor, wherein the camphor is obtained from the alpha-pinene contained in turpentine which is in turn obtained from the resinous juice that flows from maritime pine; preparing zinc camphorate from the prepared camphoric acid in the steps of: solubilizing 200 mg of the prepared camphoric acid in 1 g of ethanol at 90° C. to obtain a first mixture, adding 0.5 g of ultrapure water to the first mixture to obtain a second mixture, mixing 81.3 mg of zinc oxide with 1 g of ultrapure water by stirring to form a frying solution, pouring in the second mixture comprising camphoric acid slowly into said frying solution comprising zinc oxide while stirring intermittently in phases of stirring to obtain a third mixture, wherein in between each of the phases of stirring, heating the third mixture slightly in an oven, obtaining a precipitate of zinc camphorate and recovering it by extraction of the solvent either by heating or by freeze-drying; preparing zinc tetra-ascorbate compound from the prepared zinc camphorate by mixing the prepared zinc camphorate in a powder form to an aqueous solution of ascorbic acid to obtain a fourth mixture, wherein the molar ratio of ascorbic acid/zinc camphorate equal to 4; wherein the mixing is carried out in a dilute medium corresponding to an amount of water in a range of between 20 and 40 equivalents mL/mmol with respect to the zinc camphorate, and wherein the fourth mixture is kept under moderate stirring and under an inert atmosphere and protected from light to avoid oxidation of the ascorbic acid, until the zinc camphorate is completely dissolved to obtain zinc tetra-ascorbo-camphorate, wherein the mixing is carried out at a temperature below 25° C., wherein the inert atmosphere is maintained under argon gas, and wherein the moderate stirring is carried out for a total of 3 hours to completely dissolve zinc camphorate which reacts with the dissolved ascorbic acid to obtain a zinc tetra-ascorbo-camphorate in solution; recovering said zinc tetra-ascorbo-camphorate compound in solution into a powder form by cold freeze-drying in order to extract the solvent under vacuum and cold and the obtained zinc tetra-ascorbo-camphorate compound referred to as C14 is lyophilized and kept in a dark place to obtain lyophilized zinc tetra-ascorbo-camphorate compound referred to as C14; and mixing the lyophilized zinc tetra-ascorbo-camphorate compound referred to as C14 with a physiologically acceptable aqueous solvent to dissolve it, and at least one physiologically acceptable excipient to obtain the pharmaceutical composition for administering a therapeutically effective amount of said composition to a subject directed against viral infections caused by viruses of the family Papillomaviridae.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The accompanying drawings are included to provide a further understanding of the invention, and are incorporated in and constitute a part of the present invention and, together with the description, serve to explain the principle of the invention.

In the drawings,

FIG. 1 provides a representation of a molecule of L-ascorbic acid and a molecule of camphoric acid, and whose respective carbon atoms carrying H protons are numbered with reference to the NMR analysis ¹H of the said compound described below.

FIG. 2 provides a vertical bar graph of the results of neutralization of pseudovirions VLP-16 (also noted PsV-16) by the compound “AC” tested on Cos-7 cells, the Cos-7 cells having been pretreated with said compound “AC” 3 hours prior to inoculation of VLP-16; and compared to controls on T-cells, by measurement of the optical density OD of the luciferase-expressing pseudovirions, and as a function of the dilution of said “AC” compound.

FIG. 3 provides a vertical bar graph of the results of neutralization of VLP-16 pseudovirions (also noted as PsV-16) by the compound “AC” tested on Cos-7 cells, with inoculation of Cos-7 cells with VLP-16s being performed contemporaneously with the addition of said compound “AC”, and compared to T-cell controls, by measurement of OD optical density on luciferase-expressing PsV-16s, and as a function of dilution of said compound “AC”.

FIG. 4 provides a representation of a possible spatial structure of zinc tetra-ascorbo-camphorate referred to as C14 in aqueous medium.

FIG. 5 provides a representative graphical data for cellular viability in the presence of zinc tetra-ascorbo- camphorate referred to as C14 at different concentrations tested in a range from 1 μM to 500 μM (n=4 for each concentration) by incubating three cell lines HEC-1A (line of endocervical origin), SiHa (vaginal cells), and CaCo-2 (intestinal cells) with said concentrations of C14. The cell viability is measured after an exposure time of 24 hours using the KIT Tox-2 XTT Sigma Aldrich assay to demonstrate that C14 does not exhibit any significant change in cellular viability and thereby no toxicity to epithelial cells at concentrations less than or equal to 125 μM with respect to the no treatment (N-T) control and/or DMSO solvent control.

FIG. 6 provides a representative illustration to show the impact of zinc tetra-ascorbo-camphorate referred to as C14 on the sealing of epithelial mucosa measured in terms of the transepithelial electrical resistance (TEER) allows to evaluate the tightness of monostratified epithelial mucosa. The said assay employs specific cell culture model based on the use of cell culture inserts that have a porosity filter of 3 μm as shown and indicated in the illustration thereby allowing to mimic the apical (lumen) and basal (lamina propria) compartments, where the cell lines tested include HEC-1A and CaCo-2 for sealed monostratified epithelia, which is marked with an arrow inside the cell culture inserts which are in turn contained inside wells of a cell culture plate with basolateral compartment containing an environment of culture and inside the cell culture insert, the apical compartment contains an environment of supplemented culture where, when the cells are at confluence, the apical medium is replaced by a C14 solution of given concentration that are in a range from 1 μM to 500 μM (n=2 for each concentration).

FIG. 7 provides the graphical representation of the results with the use of the transepithelial electrical resistance (TEER) model as illustrated in FIG. 6, where the total exposure time is 24 hours. In the assay, a rupture of this resistance can lead to an increase in the transmission of the HIV virus. As seen with the demonstrated graph, zinc tetra-ascorbo-camphorate referred to as C14 of the present invention does not affect the TEER of monostratified epithelia, even at high concentrations (500 μM) with respect to the no treatment (N-T) control.

FIG. 8 provides representative graphs for the data obtained for various cytokines/chemokines namely, IL-1β, IL-8, IL-12, IFN-γ using a cytokine-chemokine multiplex assay (assays for 48 inflammation-related cytokines/chemokines by Multiplex assay (BioPlex 200 BioRad)) analysis when the cell line model, HEC-1A is incubated at confluence with zinc tetra-ascorbo-camphorate referred to as C14 at a concentration in a range from 1 μM to 500 μM (n=2 for each concentration). The results demonstrate that there is no increase in secretion by HEC-1A cells of the cytokine-chemokines assayed.

### DETAILED DESCRIPTION OF THE INVENTION

The present invention may be understood more readily by reference to the following detailed description of the invention taken in connection with the accompanying drawing figures, which forms a part of this disclosure. It is to be understood that this invention is not limited to the specific devices, medicines, systems, conditions or parameters described and/or shown herein and that the terminology used herein is for the example only, and is not intended to be limiting of the claimed invention. Also, as used in the specification including the appended claims, the singular forms ‘a’, ‘an’, and ‘the’ include the plural, and references to a particular numerical value includes at least that particular value unless the content clearly directs otherwise. Ranges may be expressed herein as from ‘about’ or ‘approximately’ another particular value. When such a range is expressed, it is another embodiment. Also, it will be understood that unless otherwise indicated, dimensions and material characteristics stated herein are by way of example rather than limitation, and are for better understanding of sample embodiment of suitable utility, and variations outside of the stated values may also be within the scope of the invention depending upon the particular application.

The terms “comprising,” “having,” “including,” and “containing” are to be construed as open-ended terms (i.e., meaning “including, but not limited to,” and permit the presence of one or more features or components) unless otherwise noted. It should be understood that while various embodiments in the specification are presented using “comprising” language, under various circumstances, a related embodiment may also be described using “consisting of” or “consisting essentially of language.

As such, the terms “a” (or “an”), “one or more,” and “at least one” can be used interchangeably herein. Furthermore, “and/or” where used herein is to be taken as specific disclosure of each of the two specified features or components with or without the other. Further, unless specifically stated or obvious from context, as used herein, the term “or” is understood to be inclusive. Also, unless specifically stated or obvious from context, as used herein, the terms “a”, “an”, and “the” are understood to be singular or plural.

It should also be understood that when describing a range of values, the characteristic being described could be an individual value found within the range. For example, “a pH from about pH 4 to about pH 6,” could be, but is not limited to, pH 4, 4.2, 4.6, 5.1, 5.5, etc. and any value in between such values. Additionally, “a pH from about pH 4 to about pH 6,” should not be construed to mean that the pH of a formulation in question varies 2 pH units in the range from pH 4 to pH 6 during storage, but rather a value may be picked in that range for the pH of the solution, and the pH remains buffered at about that pH. In some embodiments, when the term “about” is used, it means the recited number plus or minus 10% of that recited number.

By “effective amount” is meant the amount of an agent or composition as disclosed herein required to ameliorate the symptoms of a disease relative to an untreated patient. The effective amount of the active compound(s) or composition used to practice the present invention for prevention or curative treatment of a disease varies depending upon the manner of administration, the age, body weight, and general health of the subject. Ultimately, the attending physician or veterinarian will decide the appropriate amount and dosage regimen. Such amount is referred to as an “effective” amount.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the term “Virus Like Particle” (VLP) also known as pseudovirions or pseudo viruses (PsV), refers to a structure that in at least one attribute resembles a virus but which has not been demonstrated to be infectious. VLPs or PsVs in accordance with the invention do not carry genetic information encoding for the proteins of the virus-like particles. In general, VLPs or PsVs lack a viral genome and, therefore, are noninfectious. In addition, VLPs or PsVs can often be produced in large quantities by heterologous expression and can be easily purified. In this disclosure, virus pseudo particles or pseudovirions or VLPs, etc., are used interchangeably and mean the same thing.

As used herein, the term “composition” is used interchangeably with “pharmaceutical composition” and “pharmacological composition” and comprises zinc tetra-ascorbo-camphorate compound referred to as C14 which is modulated for administration via various routes of administration in a subject at the desired concentration and dilution via a suitable a physiologically acceptable aqueous solvent(s), and a suitable physiologically acceptable excipient(s) for the desired/selected route of administration in the subject.

As used herein, the term “physiologically acceptable” means solvents and excipients that can be used for administering the final composition into a human being via various, desired and selected routes of administration without causing any adverse reaction or harm to the human being.

In any of the ranges described herein, the endpoints of the range are included in the range. However, the description also contemplates the same ranges in which the lower and/or the higher endpoint is excluded. Additional features and variations of the invention will be apparent to those skilled in tire art from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such;

variations of the invention lacking limitations which have not been described herein as critical are intended as aspects of the invention.

Unless defined otherwise, all technical and scientific terms used herein have the same meaning as commonly understood by one of ordinary skill in the art to which this disclosure is related. Units, prefixes, and symbols are denoted in their Systems International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. The entire document is intended to be viewed as a unified disclosure, and it should be understood that all combinations of features described herein are contemplated. All references cited herein are hereby incorporated by reference in their entireties.

Embodiments will now be described in details with reference to the accompanying drawings. To avoid unnecessarily obscuring in the present disclosure, well-known features may not be described, or substantially the same elements may not be redundantly described, for example. This is for ease of understanding. The drawings and the following description are provided to enable those skilled in the art to fully understand the present disclosure and are in no way intended to limit the scope of the present disclosure as set forth in the appended claims.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

As provided hereinabove, there remains a problem and need for medications and methods for prevention and/or treatment of infections by a virus of the family Papillomaviridae, which is a family of non-enveloped DNA viruses whose members are known as papillomaviruses. In particular, health problems related to human papilloma virus (HPV), include genital warts and cervical cancer, and the especially oncogenic HPVs also known as high-risk HPVs are oncogenic papillomaviruses consisting of the HPV-16 genotype, and the HPV-18 genotype. To this end, the present invention provides a solution to the aforementioned problem by providing medicaments in the form of pharmaceutical compositions and methods using said pharmaceutical compositions by administering a therapeutically effective amount of said composition for preventive and/or curative treatment of viral infections caused by viruses of the family Papillomaviridae in a subject, which is a human subject.

In 2009, a Phase III trial to market the first vaginally applied HIV microbicide, a chemical barrier to protect against HIV infection during sexual intercourse, failed, calling into question any further development in this area. The group of women who received the microbicide were more contaminated than those in the control group. Later a South African research team highlighted the pro-inflammatory nature of the antiviral used. The antiviral in question caused local inflammation which in turn promoted the recruitment of immune cells in the area which favored the HIV contamination. It was therefore not the substance itself but rather the inflammation it caused that caused an increase in HIV contamination.

Most of the available antivirals at that time were known to have pro-inflammatory properties, with the exception of one that did not show measurable anti-HIV activity. Thus, another aspect of the present invention that helps prevent and/or treat against an infection involving at least one papillomavirus, particularly oncogenic papillomaviruses is to provide an antiviral as well as an anti-inflammatory compound that the present invention as disclosed herein provides to address the aforementioned problem associated with inflammation and pro-inflammatory responses in the anti-viral treatments.

Further, the present invention proposes a pharmacological composition for use in the preventive and/or curative treatment of an infection involving at least one papillomavirus, in particular at least one human papillomavirus (HPV), the said composition comprising at least one metal ascorbo-camphorate compound. Further, the present invention provides a pharmacological composition for use in the preventive and/or curative treatment of an infection involving at least one oncogenic papillomavirus, comprising said metal ascorbo-camphorate compound. In particular said infection indicates at least one oncogenic papillomavirus of the HPV-16 genotype and/or the HPV-18 genotype, where the said metal ascorbo-camphorate compound is advantageously soluble in an aqueous solvent. Also, the said metal ascorbo-camphorate compound comprises a metal ion selected from those of the group consisting of metals of the series 2 of the Mendeleev classification including Zinc, Copper, Gold, Silver, and Magnesium, where preferably said ion is a Zinc ion and more preferably it is Zinc tetra-ascorbo-camphorate also referred to as C14. The said Zinc tetra-ascorbo-camphorate compound, derived from a modified terpene, is preferably formed by Zinc camphorate and ascorbic acid, in a molar ratio of ascorbic acid to Zinc camphorate equal to 4. The C14 compound thus obtained has the advantage of being stable and of being able to be completely dissolved in an aqueous medium.

The active part of said Zinc tetra-ascorbo-camphorate compound also referred to as C14 is based on the basic terpene derivative camphorate, but the ascorbic acid part of the compound can promote the transfer of said compound through the virus itself to reach a specific target, for example its genetic material, where the active element can cause its destruction. Furthermore, the metal ion, especially in the case of Zinc, may also contribute to promote the antiviral action of said terpene derivative compound.

In accordance with one embodiment of the present invention, it discloses a method for preventive and/or curative treatment of viral infections caused by viruses of the family Papillomaviridae in a subject, said method comprising the step of administering a therapeutically effective amount of a composition to the subject, wherein the composition comprises of at least one metal ascorbo-camphorate compound, a physiologically acceptable aqueous solvent, and at least one physiologically acceptable excipient, and wherein the metal ascorbo-camphorate compound is Zinc tetra-ascorbo-camphorate referred to as C14.

In another embodiment of the method for preventive and/or curative treatment of viral infections caused by viruses of the family Papillomaviridae in a subject as disclosed herein, wherein the subject is a human.

In another embodiment of the method for preventive and/or curative treatment of viral infections caused by viruses of the family Papillomaviridae in a subject as disclosed herein, wherein the viruses are human papilloma viruses (HPV) including oncogenic papillomaviruses consisting of the HPV-16 genotype, the HPV-18 genotype, or a combination thereof.

In another embodiment of the method for preventive and/or curative treatment of viral infections caused by viruses of the family Papillomaviridae in a subject as disclosed herein, wherein the physiologically acceptable aqueous solvent is selected from a group of solvents consisting of water, a mixture of water and an alcohol, a mixture of water and another polar solvent, or a combination thereof, and wherein the alcohol or the polar solvent is physiologically acceptable. In a preferred embodiment of the present invention as disclosed herein, the physiologically acceptable alcohol in the aqueous solvent is ethanol. Further, the physiologically acceptable aqueous solvent is selected such that the Zinc tetra-ascorbo-camphorate referred to as C14 of the present invention is completely in its dissolved form in the said solvent in the composition so as to form the therapeutically effective amount of the composition to be administered to the subject as disclosed in the present invention.

In another embodiment of the method for preventive and/or curative treatment of viral infections caused by viruses of the family Papillomaviridae in a subject as disclosed herein, wherein the at least one physiologically acceptable excipient is an excipient for packaging in a galenic form of a gel or an ointment type for administering the therapeutically effective amount of a composition to the subject via the skin, and/or mucous membranes.

In another embodiment of the method of preventive and/or curative treatment of viral infections caused by viruses of the family Papillomaviridae in a subject as disclosed herein, the at least one physiologically acceptable excipient is an excipient for packaging in a solid dosage of tablets, capsules or sachets in powder form for administering the therapeutically effective amount of the composition to the subject via dissolution in water for external application and/or for ingestion orally. In this form, the solid dosage form may be for dissolution in water and/or for ingestion, for example with an aqueous liquid and may thus be tablets, capsules or sachets containing a said Zinc tetra-ascorbo-camphorate compound in powder form, which will then be swallowed with a large glass of water, or added to drinking water so as to be dissolved before the subject orally absorbs it, or it may alternatively be administered dissolved in water for use in external application such as in a patch for subcutaneous or topical delivery.

In another embodiment of the method for preventive and/or curative treatment of viral infections caused by viruses of the family Papillomaviridae in a subject as disclosed herein, the at least one physiologically acceptable excipient is an excipient for packaging in a galenic form of an ovule or a capsule or a vaginal ring or a vaginal bulb and/or pump type for administering the therapeutically effective amount of a composition to the subject via the vaginal route. This form of packaging is particularly suitable for the curative and/or preventive treatment of infections caused by HPVs, in particular those of the very frequent HPV-16 or HPV-18 genotype, which are mainly involved in sexually transmitted infections that can lead to cancers, in particular of the cervix.

In another embodiment of the method for preventive and/or curative treatment of viral infections caused by viruses of the family Papillomaviridae in a subject as disclosed herein, wherein the therapeutically effective amount of the composition comprising the Zinc tetra-ascorbo-camphorate referred to as C14 is administered to the subject in a liquid form with the Zinc tetra-ascorbo-camphorate referred to as C14 dissolved in the physiologically acceptable aqueous solvent at a dilution in a range between 1:500,000 (a five hundred thousandth) and 1:10 (a tenth), preferably in a range between 1:2,000 (a two thousandth) and 1:10 (a tenth), and more preferably in a range between 1:2,000 (a two thousandth) and 1:100 (a hundredth), and wherein the physiologically acceptable aqueous solvent is water.

In another embodiment of the method for preventive and/or curative treatment of viral infections caused by viruses of the family Papillomaviridae in a subject as disclosed herein, wherein the therapeutically effective amount of the composition comprising the Zinc tetra-ascorbo-camphorate referred to as C14 is administered to the subject in a solid form with the Zinc tetra-ascorbo-camphorate referred to as C14 at a dosage in a range between 20 milligrams (mg) and 3 grams (g) per day for a human subject, and wherein the said dosage is given as a single dose or as multiple divided doses for a cumulative dosage of a range between 20 mg and 3 g per day for the human subject.

In another embodiment of the method for preventive and/or curative treatment of viral infections caused by viruses of the family Papillomaviridae in a subject as disclosed herein, wherein the therapeutically effective amount of the composition is administered to the subject by a route of administration selected from a group consisting of intravenous route, intraperitoneal route, oral route, intranasal route, subcutaneous route, intrathecal route, intraventricular route, intramuscular route, by instillation route via a vaginal bulb and/or pump , by perfusion route, and by vaginal ring or by an imbibed sanitary pad route, and wherein the at least one physiologically acceptable excipient comprised in said composition provides the packaging for administration via the selected route of administration.

In accordance with one embodiment of the present invention, it discloses a pharmaceutical composition directed against viral infections caused by viruses of the family Papillomaviridae, the pharmaceutical composition comprising: (a) a metal ascorbo-camphorate compound; (b) a physiologically acceptable aqueous solvent; and (c) at least one physiologically acceptable excipient, wherein the metal ascorbo-camphorate compound is a metal tetra-ascorbo-camphorate, wherein the metal ascorbo-camphorate compound comprises a metal ion selected from the group comprising metals of the series 2 of the Mendeleev classification including Zinc, Copper, Gold, Silver, and Magnesium, wherein the metal ascorbo-camphorate compound is soluble in a physiologically acceptable aqueous solvent, and wherein the viruses of the family Papillomaviridae are human papilloma viruses (HPV) including oncogenic papillomaviruses consisting of the HPV-16 genotype, the HPV-18 genotype, or a combination thereof, and wherein the at least one physiologically acceptable excipient comprised in said composition provides the packaging for administration via a route of administration in a subject for a route of administration selected from a group consisting of intravenous route, intraperitoneal route, oral route, intranasal route, subcutaneous route, intrathecal route, intraventricular route, intramuscular route, by instillation route via a vaginal bulb and/or pump, by perfusion route, and by vaginal ring route.

In another embodiment of the pharmaceutical composition directed against viral infections caused by viruses of the family Papillomaviridae as disclosed herein, wherein the metal ascorbo-camphorate compound is Zinc tetra-ascorbo-camphorate referred to as C14.

In another embodiment of the pharmaceutical composition directed against viral infections caused by viruses of the family Papillomaviridae as disclosed herein, wherein the physiologically acceptable aqueous solvent is selected from a group of solvents consisting of water, a mixture of water and an alcohol, a mixture of water and another polar solvent, or a combination thereof, and wherein the alcohol or the polar solvent is physiologically acceptable. In a preferred embodiment of the present invention as disclosed herein, the physiologically acceptable alcohol in the aqueous solvent is ethanol. Further, the physiologically acceptable aqueous solvent is selected such that the Zinc tetra-ascorbo-camphorate referred to as C14 of the present invention is completely in its dissolved form in the said solvent in the composition so as to form the therapeutically effective amount of the composition to be administered to the subject as disclosed in the present invention.

In another embodiment of the pharmaceutical composition directed against viral infections caused by viruses of the family Papillomaviridae as disclosed herein, wherein the at least one physiologically acceptable excipient is an excipient for packaging in a galenic form of a gel or an ointment type for administering the therapeutically effective amount of a composition to the subject via the skin, and/or mucous membranes.

In another embodiment of the pharmaceutical composition directed against viral infections caused by viruses of the family Papillomaviridae as disclosed herein, wherein the at least one physiologically acceptable excipient is an excipient for packaging in a solid dosage of tablets, capsules or sachets in powder form for administering the therapeutically effective amount of the composition to the subject via dissolution in water for external application and/or for ingestion orally. In this form, the solid dosage form may be for dissolution in water and/or for ingestion, for example with an aqueous liquid and may thus be tablets, capsules or sachets containing a said Zinc tetra-ascorbo-camphorate compound in powder form, which will then be swallowed with a large glass of water, or added to drinking water so as to be dissolved before the subject orally absorbs it, or it may alternatively be administered dissolved in water for use in external application such as in a patch for subcutaneous or topical delivery.

In another embodiment of the pharmaceutical composition directed against viral infections caused by viruses of the family Papillomaviridae as disclosed herein, wherein the at least one physiologically acceptable excipient is an excipient for packaging in a galenic form of an ovule or a capsule or a vaginal ring or a vaginal bulb and/or pump or an imbibed sanitary pad type for administering the therapeutically effective amount of a composition to the subject via the vaginal route. This form of packaging is particularly suitable for the curative and/or preventive treatment of infections caused by HPVs, in particular those of the very frequent HPV-16 or HPV-18 genotype, which are mainly involved in sexually transmitted infections that can lead to cancers, in particular of the cervix.

In accordance with one embodiment of the present invention, it discloses a method for preparation of a pharmaceutical composition for administering a therapeutically effective amount of said composition to a subject directed against viral infections caused by viruses of the family Papillomaviridae, said method comprising the steps of: preparing camphoric acid by oxidation of camphor, wherein the camphor is obtained from the alpha-pinene contained in turpentine which is in turn obtained from the resinous juice that flows from maritime pine; preparing zinc camphorate from the prepared camphoric acid in the steps of: solubilizing 200 mg of the prepared camphoric acid in 1 g of ethanol at 90° to obtain a first mixture, adding 0.5 g of ultrapure water to the first mixture to obtain a second mixture, mixing 81.3 mg of zinc oxide with 1 g of ultrapure water by stirring to form a frying solution, pouring in the second mixture comprising camphoric acid slowly into said frying solution comprising zinc oxide while stirring intermittently in phases of stirring to obtain a third mixture, wherein in between each of the phases of stirring, heating the third mixture slightly in an oven, obtaining a precipitate of zinc camphorate and recovering it by extraction of the solvent either by heating or by freeze-drying; preparing zinc tetra-ascorbate compound from the prepared zinc camphorate by mixing the prepared zinc camphorate in a powder form to an aqueous solution of ascorbic acid to obtain a fourth mixture, wherein the molar ratio of ascorbic acid/zinc camphorate equal to 4; wherein the mixing is carried out in a dilute medium corresponding to an amount of water in a range of between 20 and 40 equivalents mL/mmol with respect to the zinc camphorate, and wherein the fourth mixture is kept under moderate stirring and under an inert atmosphere and protected from light to avoid oxidation of the ascorbic acid, until the zinc camphorate is completely dissolved to obtain zinc tetra-ascorbo-camphorate, wherein the mixing is carried out at a temperature below 25° C., wherein the inert atmosphere is maintained under argon gas, and wherein the moderate stirring is carried out for a total of 3 hours to completely dissolve zinc camphorate which reacts with the dissolved ascorbic acid to obtain a zinc tetra-ascorbo-camphorate in solution; recovering said zinc tetra-ascorbo-camphorate compound in solution into a powder form by cold freeze-drying in order to extract the solvent under vacuum and cold and the obtained zinc tetra-ascorbo-camphorate compound referred to as C14 is lyophilized and kept in a dark place to obtain lyophilized zinc tetra-ascorbo-camphorate compound referred to as C14; and mixing the lyophilized zinc tetra-ascorbo-camphorate compound referred to as C14 with a physiologically acceptable aqueous solvent to dissolve it, and at least one physiologically acceptable excipient to obtain the pharmaceutical composition for administering a therapeutically effective amount of said composition to a subject directed against viral infections caused by viruses of the family Papillomaviridae.

In another embodiment of the method for preparation of a pharmaceutical composition for administering a therapeutically effective amount of said composition to a subject directed against viral infections caused by viruses of the family Papillomaviridae as disclosed herein, wherein the physiologically acceptable aqueous solvent is selected from a group of solvents consisting of water, a mixture of water and an alcohol, a mixture of water and another polar solvent, or a combination thereof, and wherein the alcohol or the polar solvent is physiologically acceptable. In a preferred embodiment of the present invention as disclosed herein, the physiologically acceptable alcohol in the aqueous solvent is ethanol. Further, the physiologically acceptable aqueous solvent is selected such that the Zinc tetra-ascorbo-camphorate referred to as C14 of the present invention is completely in its dissolved form in the said solvent in the composition so as to form the therapeutically effective amount of the composition to be administered to the subject as disclosed in the present invention.

In another embodiment of the method for preparation of a pharmaceutical composition for administering a therapeutically effective amount of said composition to a subject directed against viral infections caused by viruses of the family Papillomaviridae as disclosed herein, wherein the at least one physiologically acceptable excipient is an excipient for packaging in a galenic form of a gel or an ointment type for administering the therapeutically effective amount of a composition to the subject via the skin, and/or mucous membranes.

In another embodiment of the method for preparation of a pharmaceutical composition for administering a therapeutically effective amount of said composition to a subject directed against viral infections caused by viruses of the family Papillomaviridae as disclosed herein, wherein the at least one physiologically acceptable excipient is an excipient for packaging in a solid dosage of tablets, capsules or sachets in powder form for administering the therapeutically effective amount of the composition to the subject via dissolution in water for external application and/or for ingestion orally. In this form, the solid dosage form may be for dissolution in water and/or for ingestion, for example with an aqueous liquid and may thus be tablets, capsules or sachets containing a said Zinc tetra-ascorbo-camphorate compound in powder form, which will then be swallowed with a large glass of water, or added to drinking water to be dissolved before the subject orally absorbs it, or it may alternatively be administered dissolved in water for use in external application such as in a patch for subcutaneous or topical delivery.

In another embodiment of the method for preparation of a pharmaceutical composition for administering a therapeutically effective amount of said composition to a subject directed against viral infections caused by viruses of the family Papillomaviridae as disclosed herein, wherein the at least one physiologically acceptable excipient is an excipient for packaging in a galenic form of an ovule or a capsule or a vaginal ring or a vaginal bulb and/or pump or an imbibed sanitary pad type for administering the therapeutically effective amount of a composition to the subject via the vaginal route. This form of packaging is particularly suitable for the curative and/or preventive treatment of infections caused by HPVs, in particular those of the very frequent HPV-16 or HPV-18 genotype, which are mainly involved in sexually transmitted infections that can lead to cancers, in particular of the cervix.

In another embodiment of the method for preparation of a pharmaceutical composition for administering a therapeutically effective amount of said composition to a subject directed against viral infections caused by viruses of the family Papillomaviridae as disclosed herein, wherein the viruses of the family Papillomaviridae are human papilloma viruses (HPV) including oncogenic papillomaviruses consisting of the HPV-16 genotype, the HPV-18 genotype, or a combination thereof.

In accordance with one embodiment of the present invention, it discloses a pharmaceutical composition for use in the preventive and/or curative treatment of viral infections caused by viruses of the family Papillomaviridae in a subject, the pharmaceutical composition comprising: (a) a metal ascorbo-camphorate compound; (b) a physiologically acceptable aqueous solvent; and (c) at least one physiologically acceptable excipient, wherein the metal ascorbo-camphorate compound is Zinc tetra-ascorbo-camphorate referred to as C14, wherein the metal ascorbo-camphorate compound is soluble in a physiologically acceptable aqueous solvent, wherein the physiologically acceptable aqueous solvent is selected from a group of solvents consisting of water, a mixture of water and an alcohol, a mixture of water and another polar solvent, or a combination thereof, and wherein the alcohol or the polar solvent is physiologically acceptable, wherein the viruses of the family Papillomaviridae are human papilloma viruses (HPV) including oncogenic papillomaviruses consisting of the HPV-16 genotype, the HPV-18 genotype, or a combination thereof, and wherein the pharmaceutical composition is administered to the subject by a route of administration selected from a group consisting of intravenous route, intraperitoneal route, oral route, intranasal route, subcutaneous route, intrathecal route, intraventricular route, intramuscular route, by instillation route via a vaginal bulb and/or pump, by perfusion route, and by vaginal ring route, and wherein the at least one physiologically acceptable excipient comprised in said composition provides the packaging for administration via the route of administration. In a preferred embodiment of the present invention as disclosed herein, the physiologically acceptable alcohol in the aqueous solvent is ethanol. Further, the physiologically acceptable aqueous solvent is selected such that the Zinc tetra-ascorbo-camphorate referred to as C14 of the present invention is completely in its dissolved form in the said solvent in the composition so as to form the therapeutically effective amount of the composition to be administered to the subject as disclosed in the present invention.

In another embodiment of the pharmaceutical composition for use in the preventive and/or curative treatment of viral infections caused by viruses of the family Papillomaviridae in a subject as disclosed herein, wherein the at least one physiologically acceptable excipient is an excipient for packaging in a galenic form of a gel or an ointment type for administering the therapeutically effective amount of a composition to the subject via the skin, and/or mucous membranes.

In another embodiment of the pharmaceutical composition for use in the preventive and/or curative treatment of viral infections caused by viruses of the family Papillomaviridae in a subject as disclosed herein, wherein the at least one physiologically acceptable excipient is an excipient for packaging in a solid dosage of tablets, capsules or sachets in powder form for administering the therapeutically effective amount of the composition to the subject via dissolution in water for external application and/or for ingestion orally. In this form, the solid dosage form may be for dissolution in water or for ingestion, for example with an aqueous liquid and may thus be tablets, capsules or sachets containing a said Zinc tetra-ascorbo-camphorate compound in powder form, which will then be swallowed with a large glass of water, or added to drinking water to be dissolved before the subject orally absorbs it, or it may alternatively be administered dissolved in water for use in external application such as in a patch for subcutaneous or topical delivery.

In another embodiment of the pharmaceutical composition for use in the preventive and/or curative treatment of viral infections caused by viruses of the family Papillomaviridae in a subject as disclosed herein, wherein the at least one physiologically acceptable excipient is an excipient for packaging in a galenic form of an ovule or a capsule or a vaginal ring or a vaginal bulb and/or pump or an imbibed sanitary pad type for administering the therapeutically effective amount of a composition to the subject via the vaginal route. This form of packaging is particularly suitable for the curative or preventive treatment of infections caused by HPVs, in particular those of the very frequent HPV-16 or HPV-18 genotype, which are mainly involved in sexually transmitted infections that can lead to cancers, in particular of the cervix.

In a preferred embodiment of the present invention, the pharmaceutical composition for use in the preventive and/or curative treatment of an infection involving at least one papillomavirus as disclosed herein, comprises zinc tetra-ascorbo-camphorate referred to as C14, which has been obtained by a preparation process in which ascorbic acid is mixed with zinc camphorate by adding the zinc camphorate in powder form to an aqueous solution of ascorbic acid in a molar ratio of ascorbic acid/zinc camphorate equal to 4, the mixture being carried out in a dilute medium corresponding to a quantity of water of between 20 and 40 equivalents mL/mmol, in particular between 20 and 30 equivalents mL/mmol, relative to the zinc camphorate, the mixture being kept under stirring, and preferably under an inert atmosphere and protected from light, until the zinc camphorate is completely dissolved. The said zinc tetra-ascorbo-camphorate compound can then be recovered in solid form by extracting the aqueous part preferably cold and under vacuum.

In a preferred embodiment of the present invention as disclosed herein, the ascorbic acid is preferably L-ascorbic acid. Advantageously according to the invention, the zinc tetra-ascorbo-camphorate thus obtained was then collected by lyophilization.

Optionally but preferably, in the present invention as disclosed herein, the zinc camphorate is prepared in a preliminary step from camphoric acid solubilized in a polar solvent based on ethanol or 90° alcohol, to which a little water is added, and this camphoric solution is added to a milky aqueous solution of zinc oxide in a camphoric acid/zinc oxide molar ratio equal to 1/1, and the zinc camphorate precipitate is recovered by extraction of the solvent.

The present invention as disclosed herein also relates to a medicament comprising a metal ascorbo-camphorate compound as described above for use in the preventive and/or curative treatment of viral infections involving at least one papillomavirus, in particular a human papillomavirus (HPV), and more particularly an oncogenic papillomavirus, in particular of the HPV-16 genotype or the HPV-18 genotype.

The present invention as disclosed herein also relates to a method for preventive and/or curative treatment of viral infections involving at least one papillomavirus, in particular a human papillomavirus (HPV), and more particularly an oncogenic papillomavirus, in particular of the HPV-16 genotype or of the HPV-18 genotype, in which a composition comprising a metal ascorbo-camphorate compound is administered, as described above. The invention relates to a method for preventive and/or curative treatment of said viral infections comprising administering, advantageously by topical application, to a subject in need thereof a preventively or curatively effective amount of a compound ascorbo-metal-camphorate compound as described or a composition comprising said ascorbo-metal-camphorate compound as described above.

The present invention as disclosed herein also relates to a process for the preparation of a pharmaceutical composition for use in a method for the preventive and/or curative treatment of viral infections involving at least one papillomavirus, in particular a human papillomavirus (HPV), said process being as described above. For more details on the manufacture of a said ascorbo-metal-camphorate compound, reference may also be made to patent application FR2908660, in which preparations of terpene derivative compositions, in particular ascorbo-metal-camphorate compounds, usable as an anti-microorganism agent, are described, but no use for the treatment of infections involving papillomaviruses, non-enveloped viruses, is described.

The invention will be further explained by the following Examples, which are intended to purely exemplary of the invention, and should not be considered as limiting the invention in any way.

### EXAMPLES

### Example 1

### Preparation of a Zinc Tetra-Ascorbo-Camphorate Compound Also Referred to as C14 as Disclosed Herein

An experimental preparation of the zinc tetra-ascorbo-camphorate compound also referred to as C14 is provided herein which was prepared based on ascorbic acid, camphoric acid and zinc was carried out under ambient conditions of temperature and pressure, i.e., approximately 298.15 ° K and 10° Pa. Nevertheless, in some phases the mixtures were slightly heated and stirred. The process as disclosed herein was started from camphor. Camphor is generally obtained from the alpha-pinene contained in turpentine, which is obtained from the resinous juice that flows from maritime pine. Thus, alpha-pinene was converted into camphor by passing through various intermediates, including bornyl chloride, camphene and the organic ester of isoborneol. Camphoric acid of the following chemical formula referred to as [Chem 1] was prepared by oxidation of camphor:

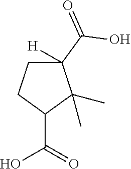

Next, Zinc camphorate was prepared starting with 200 mg of camphoric acid as prepared hereinabove which was then solubilized in 1 g of ethanol at 90° diluted with water. Then, 0.5 g of ultrapure water was added to the abovementioned first mixture. Then the said first mixture of camphoric acid was combined with zinc oxide in solution. For this purpose, 81.3 mg of zinc oxide was mixed with 1 g of ultrapure water in a second mixture which, after stirring, formed a frying solution. The abovementioned first mixture of camphoric acid was then slowly poured into the milky solution of the said frying solution. The whole of the so forming third mixture was stirred moderately and intermittently. In addition, between each stirring phase, the forming third mixture was slightly heated, for example in the oven, in order to accelerate the reaction. A precipitate, zinc camphorate, was then formed and recovered by extraction of the solvent, either by heating or by freeze-drying.

The zinc tetra-ascorbate compound was then prepared as follows: 288 mg (1.09 mmol, 1 eq.) of the previously obtained zinc camphorate was mixed with a 22 mL (20 eq.) solution of pure water containing 770 mg (4.38 mMol, 4 eq.) of L-ascorbic acid of the following chemical formula referred to as [Chem 2]:

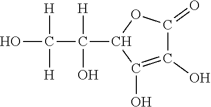 [SMILES: O=C1OC(C(O)CO)C(O)=C1O]

In the example, preferably, the mixing was carried out at a temperature below 298.15 ° K (i.e., 25° C.), and moderately stirred, in an inert atmosphere (under argon gas) and protected from light in order to avoid oxidation of the ascorbic acid. After one hour, 10 mL (10 eq.) of water was added to complete the dissolution of the zinc camphorate in the third mixture, leaving the said mixture to stir moderately for another 2 hours. Quantities expressed as “eq.” are molar equivalents or mL/mmol relative to zinc camphorate.

Further, during the intermittent stirring for the forming third mixture, for a total of 3 hours, all of the zinc camphorate was dissolved in solution and reacted with the dissolved ascorbic acid to form a zinc tetra-ascorbo-camphorate compound in solution. The said dissolved zinc tetra-ascorbo-camphorate compound was then recovered in powder form by cold freeze-drying, i.e., by extracting the solvent (water) under vacuum and cold. Notably, recovery by heating and evaporation of the solvent should be avoided to prevent oxidation of the ascorbic acid.

Further, the resulting prepared zinc tetra-ascorbo-camphorate compound also referred to as C14 was lyophilized and kept in a dark place. The said zinc tetra-ascorbo-camphorate compound or C14 will be referred to simply as “ascorbo-camphorate” or as “AC” in the following description of the example.

The molecular ratio of the ascorbic acid portion of the compound to the camphorate portion was determined, by ¹H proton NMR analyses. These ¹H proton NMR analyses of the recovered powder as prepared by the method as disclosed hereinabove in this example provided that combining four moles of ascorbic acid to one mole of zinc camphorate lead to a stable compound, the zinc tetra-ascorbo-camphorate compound also referred to as C14 with a signal around 4.65 ppm which is typical for it.

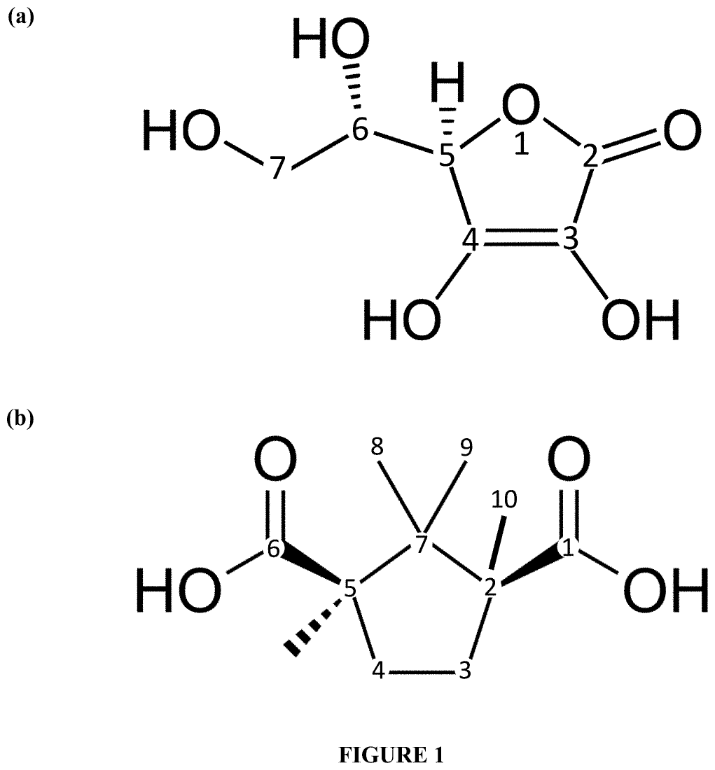

As illustrated in the FIG. 1 as disclosed herein, it represents a molecule of L-ascorbic acid and a molecule of camphoric acid, the respective atoms of which, in particular the carbon atoms carrying H protons, are numbered with reference to the ¹H NMR analysis of the said compound described below. Further, as disclosed herein, the NMR spectra were performed on a BRUCKER Avance DPX-300 spectrometer (7.05 Tesla magnet) equipped with a 5 mm diameter QNP 1H/13C/31 probe with Z gradient and lock ²H. The ¹H proton NMR analysis was conducted on a solution of 15 mg of product in 500 μL of D20 (99.8% deuterated water).

Table 1 lists the chemical shifts of the protons ¹H (in ppm) for samples of camphoric acid and L-ascorbic acid alone, and the resulting zinc tetra-ascorbate compound, studied by proton NMR ¹H at 27° C., the atom numbers indicated in the first column of the table corresponding respectively to a carbon atom linked to the proton under consideration, according to the numbering given on the structures of the respective molecules of ascorbic acid (movements in the second column of the table) and of camphoric acid (shifts in the third column of the table) as shown in FIG. 1. The displacements for the compound “AC” are listed in the fourth column of the following Table 1 in relation to the atoms from the ascorbic acid and camphoric acid molecules.

| N° | δppm | δppm | ppm |
|---|---|---|---|
| of | Ascorbic | Camphoric | “AC” or C14 |
| atoms | Acid | Acid | Compound |
| 5 | 4.85, d, 1H | — | 4.65 |
| 6 | 3.95, td, 1H | — | 3.95 |
| 7 | 3.64, d, 2H | — | 3.65 |
| — | — | — | — |
| 5 | — | 2.83, dd, 1H | 2.78 |
| 3 | — | 2.31, d, d, d, 1H | 2.30 |
| 4 | — | 1.82, m, 2H | 1.82 |
| 3 | — | 1.46, d, d, d, 1H | 1.44 |
| 10 | — | 1.17, s, 3H | 1.14 |
| 8 | — | 1.14, s, 3H | 1.11 |
| 9 | — | 0.76, s, 3H | 0.73 |

As mentioned in the Table 1 hereinabove,
s: singlet;
d: doublet;
dd: doublet of doublet;
ddd doubles of doublet of doublet;
td: triplet of doublets.

The signal around 4.65 ppm is typical of the “AC” compound.

The number of ascorbic acid molecules interacting with a zinc camphorate molecule, i.e., the molar ratio of ascorbic acid to camphorate, can be checked by calculating an integration ratio by comparing the integration value corresponding to the H proton of ascorbic acid giving a signal at 3.9 s ppm, integrating for a proton (single proton carried by carbon number 6 of ascorbic acid as shown in FIG. 1, to the integration value of the methyl group giving a signal around 0.73 ppm of the camphorate integrating for three protons (protons carried by carbon number 9 of camphoric acid in FIG. 1)).

Thus, taking into account the experimental uncertainties, when the ascorbic acid/camphorate molar ratio was determined by NMR analysis of the proton ¹H and calculation of the said integration ratio (H at 3.95 ppm of ascorbic acid; 3H of camphorate at about 0.73 ppm) as described above, the compound obtained is considered to have an ascorbic acid/camphorate molar ratio equal to 4, and therefore to be compliant, if this integration ratio is between about 3.5 and 4.5.

Various tests in conjunction with ¹H proton NMR analyses as indicated above further showed that the synthesis of compound “AC” during the mixing of ascorbic acid with zinc camphorate was favored in a relatively dilute medium, i.e., preferably for a volume of water of between 20 and 40 equivalents mL/mmol, in particular between 20 and 30 equivalents mL/mmol, with respect to zinc camphorate. The following was then obtained directly, without the need for any subsequent purification steps, resulting in a stable zinc tetra-ascorbo-camphorate compound by simply reacting four moles of ascorbic acid to 1 mole of zinc camphorate.

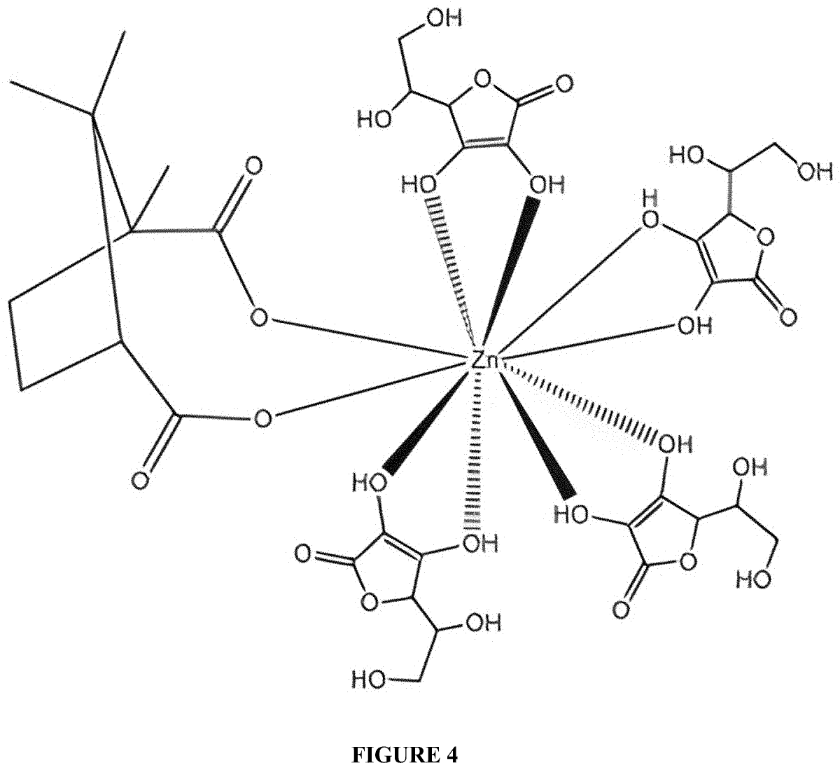

The crude formula for zinc tetra-ascorbate is 4(C₆H₆O₆)Zn(C₁₀H₁₄O₄). However, the compound can form a number of hydrates. The FIG. 4 as disclosed herein represents an illustration of a possible spatial structure of zinc tetra-ascorbo-camphorate also referred to as C14 in aqueous medium.

### Example 2

### Cytotoxicity of the Resulting Zinc Tetra-Ascorbo-Camphorate Compound Also Referred to as C14 as Disclosed Herein

In this example, the cytotoxicity of the experimental zinc tetra-ascorbo-camphorate compound also referred to as C14 was monitored using Sigma's MTT assay. The cytotoxicity study was conducted on three types of cells: on Vero cells, on human diploid MRC5 cells (human fibroblast and embryonic type), and on Cos-7 cells. Cultures of these cells were treated with compositions containing said zinc tetra-ascorbo-camphorate compound or C14 referred to simply as “ascorbo-camphorate” or as “AC” in solution in water at different dilution ratios and the MTT reagent was then added to each of the solutions. The latter forms crystals with the highly metabolic living cells.

The absorbance at 490 nm of the dissolved crystals corresponds to the number of living cells. By comparison with cultured but untreated cells of the same cell line, a survival percentage is obtained, revealing the toxicity of the said ascorbo-camphorate compound. For this purpose, a stock solution having a molarity of 0.05155 M was made by dissolving 0.5 g of said ascorbic acid camphorate obtained above in 10 ml of water. The said stock solution is diluted at different rates, from one-tenth to one-billionth so that a series of diluted solutions are obtained having respective molarities between 5.15.10⁻⁵ μM and 5155 μM.

In a first series of tests, the Cytotoxicity of said ascorbo-camphorate on Vero cells was verified with these solutions at dilution rates ranging from one tenth to one billionth. Vero cell viability tests showed that the percentage of living cells remaining was greater than or equal to 50% for a 50-fold dilution. The solutions tolerable by Vero cells were therefore those with a dilution rate between one fiftieth and one billionth (value included), or in other words, solutions with a molarity of the said ascorbo-camphorate between 5.15.10⁻⁵ μM (value included) and 1031 μM (value excluded).

In a second series of tests, the cytotoxicity of said ascorbo-camphorate on MRC5 cells was tested with the solutions at dilution ratios ranging from 0 to 100 millionths. Viability tests on MRC5 cells showed that the percentage of living cells remaining was greater than or equal to 50% at one-tenth dilution. The solutions tolerable by MRC5 cells were therefore those with a dilution rate between one tenth and 100 millionths (inclusive value), or in other words solutions with a molarity of the said ascorbo-camphorate between 5.15.10-4 μM (inclusive value) and 5155 μM (exclusive value).

In a third series of tests, the cytotoxicity of said ascorbo-camphorate on Cos-7 cells was verified. High concentrations of said ascorbo-camphorate may be required to produce an effective formulation. Therefore, the intrinsic toxicity of concentrations of said ascorbo-camphorate up to 5,000 μg/mL was evaluated using a colorimetric cell viability assay. Cos-7 cells were exposed to serial dilutions of the stock solution of said ascorbo-camphorate for 24 hours.

The viability index, or the fraction of viable cells after microbicide treatment relative to the fraction of viable cells exposed to the control condition, was calculated. Cells treated with 0.1% PBS-azide solution were used as a positive control for toxicity. The viability index, or the fraction of viable cells after treatment with the compound “AC” relative to the fraction of viable cells exposed to the natural state, was calculated. The said zinc tetra-ascorbo-camphorate exhibited viability indices of 0.8 to 1.1 at all concentrations tested, indicating that it is largely non-toxic. The 50% cytotoxic concentration (CC50) of said ascorbic camphorate was calculated using a dose-response-inhibition analysis on GraptiPad Prism v5.04 software (from GraphPad Software). It was estimated to be greater than 0.00515 M.

### Example 3

### Pre-Clinical Evaluation of the In Vitro Antiviral Activity of the Experimentally Obtained Zinc Tetra-Ascorbate Also Referred to as C14 as Disclosed Herein Against HPV-16 Virus

Experiment to evaluate the antiviral activity of said terpene derivative compound zinc tetra-ascorbate also referred to as C14 as disclosed herein is referred to simply as “ascorbo-camphorate” or as “AC” in this example. This experiment provides the data and analysis of an in vitro “AC” experimental treatment against the HPV-16 papillomavirus which was carried out by neutralizing the entry of viral pseudo particles (VLP-16) of the HPV-16 virus which was inoculated into Cos-7 cells, pretreated or treated contemporaneously to the inoculation of the VLP-16 with solutions of increasing concentrations of the said ascorbo-camphorate compound (AC).

The above-described aqueous solutions of said ascorbo-camphorate compound having tolerable cytotoxicity, having a dilution ratio of between 1/10² or 1:100 (one hundredth) and 1/10⁸ or 1:100000000 (100 millionths). That is to say, having a molarity of said ascorbo-camphorate of between 5.15.10−⁴ μM (inclusive value) and 515.5 μM (inclusive value), was retained for this assessment.

Since HPV-16 viruses are not culturable, it is known to use pseudovirions or VLPs to test potential antiviral agents. The methodology for producing these vectors is known per se, and is not the subject of the present invention.

The production of the aforementioned VLPs can for example be performed as described in the prior art [see for example, Buck C B, Thompson C D. Production of papillomavirus-based gene transfer vectors. Curr Protocol Cell Biol. 2007 December; Chapter 26: Unit 26.1.] using the protocol for the production of papillomavirus-based vectors (pseudovirions or VLPs), published by the Laboratory of Cellular Oncology, National Cancer Institute, National Institute of Health, Rockville Pike, Bethesda, Md., USA, (see for example the website: https://ccrod.cancer.gov/confluence/display/LCOTF/PseudovirusProduction).

Briefly, according to the abovesaid protocol, 293TT cells were transfected with each of the plasmid vectors that formed the L1/L2 structural proteins, and incubated for 72 hours at +37° C. The cells were then lysed in buffer (0.5% Nonidet™ P 40 Substitute/1×PBS, Sigma-Aldrich, St. Louis, Mo., USA) before being sonicated three times for 15-second cycles to break the nuclear membrane and release the VLPs. The resulting nuclear extracts were purified by ultracentrifugation at 30,500 rpm (SW 32 Ti Swinging-Bucket rotor, Beckman Coulter, Inc., Brea, Calif., USA) for 26 h at +4° C. in a cesium chloride (CeCl) gradient. The fraction containing VLPs was recovered and diluted in 300 μL of Dulbecco's phosphate-buffered saline (DPBS) (Thermo Fisher Scientific, Waltham, Mass., USA) and stored at +4° C. before being used for serological analyses. Pseudovirions can also be purchased directly from laboratories known in the trade.

In the case of the present tests in this example, pseudovirions were produced at the Université François Rabelais de Tours (France), under the direction of Professor A. Touzet. In brief, the VLP-16 virus pseudo particles or pseudovirions or VLPs were produced by transfection of purified plasmids containing the L1 and L2 structural proteins of the HPV-16 capsomers, and carrying a gene encoding the luciferase marker gene, into cell culture of the 293-cell line. VLP-16s were collected by nuclear lysis, with purification by CeCl gradient ultracentrifugation.

The positive control for the neutralization assay as disclosed herein used purified VLP-16 inoculum pre-incubated with purified antibodies against HPV (incubation for 1 hour of said VLP nuclear lysate in a solution of anti-HPV antibodies in DMED culture medium). These anti-HPV antibodies served as a positive control in the plaque assay for neutralization of Cos-7 cell infection by VLP-16.

Cells of the Cos-7 cell line were cultured in 96-well microplates, 5% CO₂ and 98% H₂O, at 37° C. Inoculation of Cos-7 cells was performed as follows: the inoculum mixture was added to the wells containing the confluent Cos-7 sheets, incubated for 72 hours at 37° C., 5% CO₂ and 98% H₂O, and the supernatant is washed and discarded from the mixture.

Cos-7 cells that may have internalized the luciferase-expressing VLPs were then recovered. The Cos-7 cells were then lysed; then the substrate (luciferin) of the reporter gene (luciferase) was added with dark incubation, for reading using a luminometer.

The effectiveness of the treatment was revealed by luminometry (measurement of optical density, due to the bioluminescence reaction caused by luciferase, which is known per se). The absorbance (optical density) decreased in proportion to the efficiency of the neutralization by the ascorbo-camphorate compound of the present invention also referred to as C14 herein.

Positive controls consisted of: (i) wells with Cos-7 that was exposed to VLP-16 without pre-incubation with the active ingredient to be tested, and (ii) a lectin-like molecule from the laboratory known to inhibit infection of Cos-7 cells by VLPs.

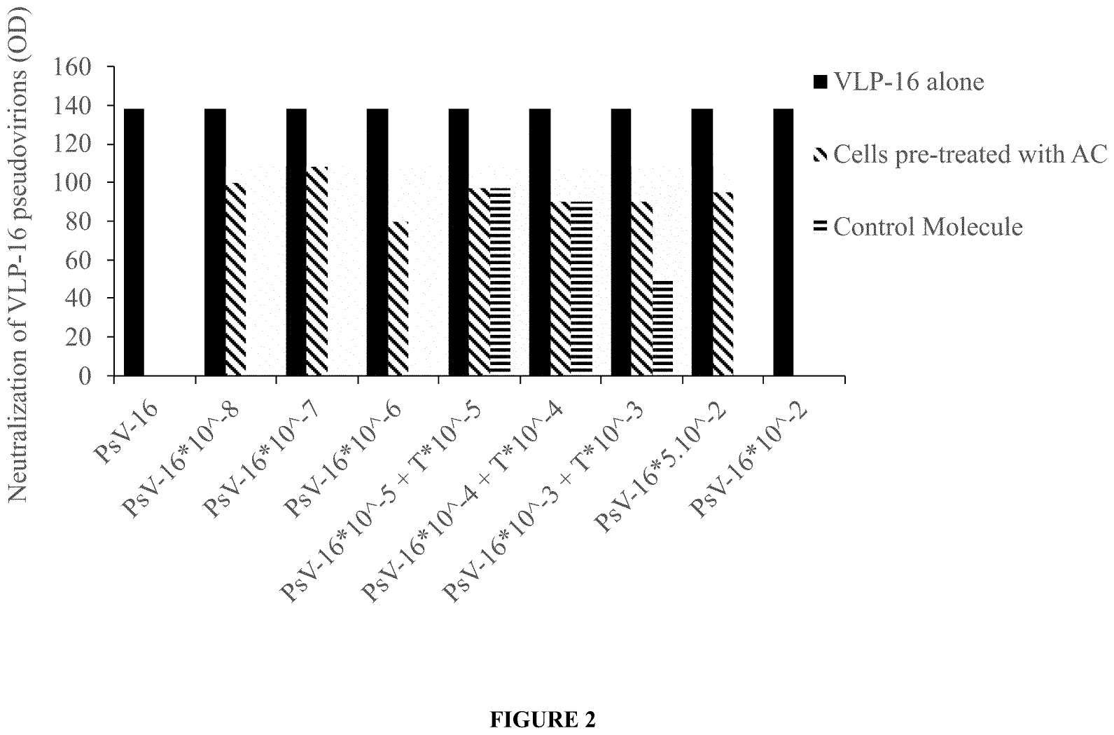

FIG. 2 as disclosed herein provides a representative vertical bar graph showing the results of the aforementioned neutralization of VLP-16 pseudovirions (also noted as PsV-16) by the compound “AC” or C14 as referred to herein as tested on Cos-7 cells, the Cos-7 cells having been pretreated with said compound “AC” 3 hours prior to VLP-16 inoculation; and compared to controls on T cells, by measurement of OD on luciferase-expressing pseudovirions, and as a function of dilution of said compound “AC”. It is noted that for clarity of reading on the diagram, the dilutions of the compound “AC” are written with the inverse of the quantile of the dilution rate, for example a dilution to the hundredth is written 10⁻² or specifically as 10\hat{ }−2. The dilution ratios of the compound “AC” solution range from 1/10⁸ as 10\hat{ }−8 to 1/10² as 10\hat{ }−2 as shown on the x-axis labels of the graph in FIG. 2. As demonstrated with FIG. 2, the inhibition of VLP-16 internalization by the ascorbo-camphorate compound includes a plateau phase characterized by a rather weak but constant neutralizing effect, with the increase of the concentration of the ascorbo-camphorate compound in the medium, then a total inhibition of VLP-16 internalization was observed at the dilution of the ascorbo-camphorate compound of 1/10² (i.e., a molarity of 515.5 μM in compound “AC”). These results clearly show that the plaque neutralization of Cos-7 cells worked very well (positive lectin inhibitor control). Upon pretreatment of Cos-7 cells with the compound “AC” prior to VLP-16 inoculation, the said compound “AC” inhibited the penetration of VLP-16 into Cos-7 cells by 30-40% as compared to the controls of Cos-7 cells infected with VLP-16 not treated with the compound “AC”.

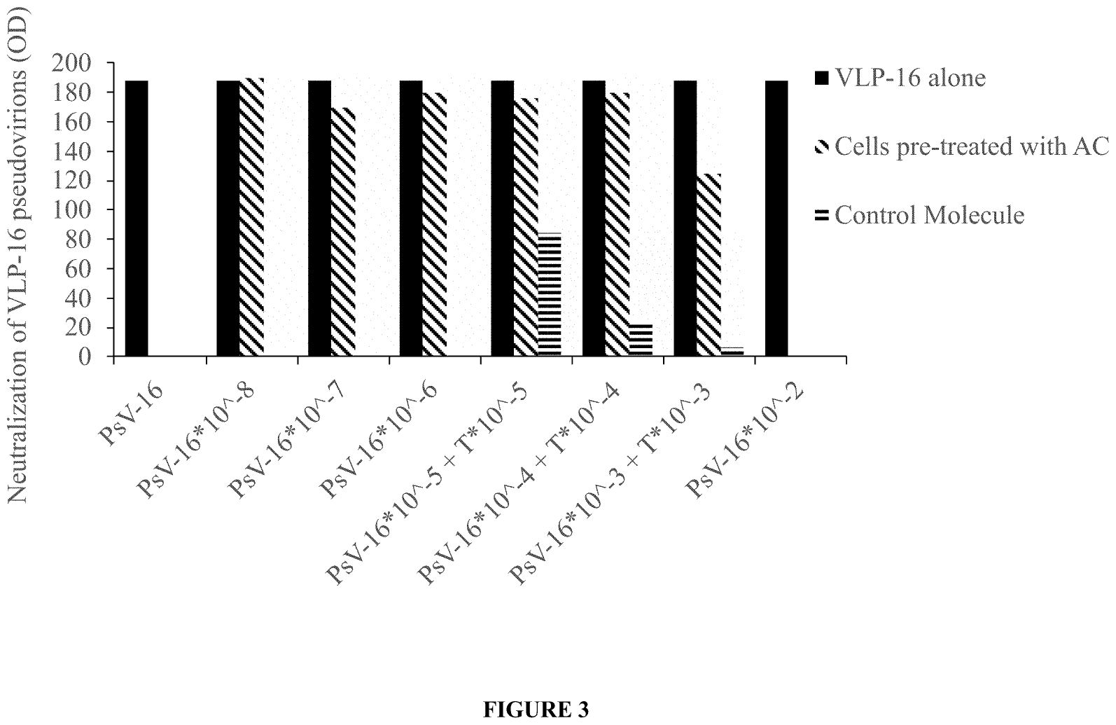

FIG. 3 as disclosed herein provides a vertical bar graph of the results of neutralization of VLP-16 pseudovirions (also noted as PsV-16) by the compound “AC” tested in Cos-7 cells, inoculation of Cos-7 by the VLP-16s being carried out contemporaneously with the addition of said “AC” compound, and by comparison with controls on T cells, by measurement of the optical density OD on the PsV-16s expressing luciferase, and as a function of the dilution of said “AC” compound. It is noted that for clarity of reading on the diagram, the dilutions of the compound the numbers “AC” was written with the inverse of the dilution ratio, e.g., a one-hundredth dilution is written as 10⁻² or specifically as 10\hat{ }−2. The dilution ratios of the of the “AC” compound goes from 1/10⁸ as 10\hat{ }−8 to 1/10² as 10\hat{ }−2 as shown on the x-axis labels of the graph in FIG. 3. As demonstrated with FIG. 3, the results there show that inoculation of Cos-7 cells with VLP-16s contemporaneously with the addition of the compound “AC” achieved up to 33% inhibition of VLP-16 penetration into Cos-7 cells, compared to controls of Cos-7 cells infected with VLP-16s not treated with the compound “AC”, with the 33% being achieved at a concentration of the said ascorbo-camphorate compound at the dilution of 1:10³ (molarity of 51.5 μM in compound). Total inhibition of VLP-16 internalization is observed at the 1/10 dilution of the ascorbo-camphorate compound (i.e., a molarity of 515.5 μM in compound “AC”). This inhibition by addition of the compound “AC” contemporaneously with inoculation appeared to be less important than when Cos-7 cells were pre-treated with the compound “AC” before inoculation with VLP-16, suggesting a very early inhibition mechanism of the penetration of virus pseudo particles into Cos-7 cells.

Further pre-clinical testing was conducted to confirm the in vitro antiviral activity of the zinc tetra-ascorbo-camphorate compound referred to as C14 as obtained in the present invention disclosed herein, against HPV-16. The assays were conducted in a manner similar to that described above with pseudovirions: one set of assays was performed by pretreating Cos-7 cells with zinc tetra-ascorbo-camphorate three hours prior to inoculation of the VLP-16 pseudovirions, and another set of assays was performed by adding zinc tetra-ascorbo-camphorate contemporaneously with the inoculation of the pseudovirions to the Cos-7 cells. For each series, the experiments were repeated three times.

To assess pseudovirions infectivity, linear reporter gene expression and to perform titration of pseudovirions solutions, Cos-7 cells were grown (10⁴ cells/well) in supplemented DMEM in 96-well plates (Thermo Fisher Scientific) in 5% CO₂ at +37° C. until cell confluence was 40-50%. Cells were transduced by replacing the culture medium with fresh unsupplemented DMEM containing serial dilutions of the previously produced PsVs stock solutions and incubated at +37° C. One well containing 1 μg of the pGL4.10[luc2] vector, encoding the luciferase reporter gene, was made as a positive control and another was the cell control for background luminescence (without PsVs or reporter plasmid). After a 3 hours incubation period, 100 μL of growth medium was added to each well and the transduced Cos-7 cells were incubated for 48 h at +37° C. in 5% CO₂. Then, the growth medium was removed and the transduced Cos-7 cells were washed with DPBS and incubated in the dark for 15 min in 100 μL of Pierce™ Firefly Luciferase One-Step Glow Assay (Thermo Fisher Scientific). The resulting cell lysates were harvested and transferred to white, 96-well microplates and luciferase enzyme activity was measured using the Luminoskan Ascent brand luminometer (from Thermo Fisher Scientific) after adding 50 μL of luciferin substrate to each well. Dilutions of pseudovirions that gave at least 80% of the luminescence of the positive control after adjustment with background luminescence were selected as suitable and therefore chosen as working dilutions for the neutralization experiments.

Serial dilutions of the stock solution of compound “AC” covering an appropriate concentration range were subjected to an HPV inhibition assay based on VLP-16 pseudovirions. Cos-7 cells were pre-plated (10⁴ cells/well) in DMEM supplemented in 96-well plates and incubated for 24 hours in 5% CO₂ at +37° C.

In the first set of assays, Cos-7 cells were pre-incubated for 3 hours in the “AC” dilution series (“AC” diluted in un-supplemented DMEM). After the pre-incubation time, the “AC” dilutions were removed and replaced with VLP-16 pseudovirions diluted in un-supplemented DMEM.

In the second set of assays, the stock solution of “AC” and the stock solution of VLP-16 pseudovirions were mixed together in the same tube with un-supplemented DMEM until the desired concentration of “AC” and VLP-16 pseudovirions in the inoculum was reached (100 μL). Infected cells were grown overnight at +37° C. and then fed with 100 μL of supplemented DMEM. After an additional 24 h of growth at +37° C., the medium was removed and the cells were washed with DPBS and incubated in the dark for 15 min in 100 μL of Pierce™ Firefly Luciferase One-Step Glow Assay (from Thermo Fisher Scientific). Cell lysates were harvested and transferred to a white, 96-well microplate and luciferase enzyme activity was measured as previously described. A negative control well (no luminescent signal inhibition) containing 1 μg of pGL4.10[luc2], and a well containing only Cos-7 cells (without pseudovirions or reporter plasmid) for background luminescence control, were made up. Inhibition of the luminescence signal by more than 80% was considered an effective inhibition of pseudovirions transduction in Cos-7 cells. A pool of sera from individuals who received three doses of the HPV vaccine marketed under the trade name Gardasil-9® (from Merck & Co. hic.) was the positive control for the VLP-16 pseudovirion-based inhibition ballast (>80% luminescence signal inhibition).

For these assays, the inhibitory concentration 50 (IC50), corresponding to the concentration of the compound “AC” that induces a 50% inhibition of the activity of VLP-16 pseudovirions on Cos-7 cells, was calculated using a dose-response-inhibition analysis on GraphPad Prism v5.04 software (from GraphPad Software). Therapeutic indices (TI=GC50/IC50) were calculated, the cytotoxic concentration CC50 having been determined during cytotoxicity assays on Cos-7 cells.

For the series of assays with pre-treatment of Cos-7 cells with the compound “AC”, the inhibitory concentration 50 (IC50) was estimated to be 2.8 μg/mL (2.9 μM) of “AC”, and the therapeutic indices TI ranged from >960 to >3,330.

For the series of assays where pseudovirions were added simultaneously to serial dilutions of

“AC”, the estimated inhibitory concentration 50 (IC50) is 8.1 μg/mL (8.3 μM) of “AC” and the therapeutic indices TI range from >410 to >820.

Pre-treatment of Cos-7 cells with the “AC” compound prior to the addition of pseudovirions was associated with more potent anti-HPV activity than the simultaneous use of the “AC” compound and pseudovirions, confirming that the “AC” compound would act in the early stages of viral infection by preventing the attachment of HPV to binding sites in the target cells, rather than acting directly on viral post adsorption events.

The spatial configuration of the compound “AC” (refer to FIG. 4), is likely to modify the specific recognition of the virus with its host cell during the first stage of infection. Furthermore, based on the inhibition assays, it is also possible to deduce that the aqueous composition of the compound “AC” may interact with the L1 and L2 proteins of the HPV-16 pseudovirions and influence their ability to bind to Cos-7 cells, as the four high polarity ascorbic acid moieties probably allow the molecule to be solubilized in aqueous or physiological environments, providing Zn²⁺-camphorate or hydrated zinc-camphorate species, which are highly reactive with proteins.

This property of strong inhibition of the adsorption of VLP-16 pseudovirions when the “AC” compound is applied in anticipation of exposure to pseudovirions confirms the usefulness of this compound, especially by topical application, to prevent the acquisition of HPV in sexually exposed individuals, especially women.

According to the invention, the stability of said zinc tetra-ascorbo-camphorate compound as disclosed and demonstrated in the present invention makes it possible to provide a composition which comprises a reliable and repeatable active amount of the active principle, in particular of the major principle which is based on the camphorate.

The present disclosure and invention thus meet the long-standing need for a non-vaccine composition for preventive and/or curative treatment of HPV infections.

### Example 4

### Assessment of Cell Viability

In this example, the cell viability as a measure of non-toxicity was assessed using KIT Tox-2 XTT Sigma Aldrich, where cell viability was measured in terms of the ability of cells to degrade a colorless compound into a colored compound (mitochondrial enzymes) using color intensity as being proportional to the cell viability. Three cell lines as models were used including HEC-1A (line of endocervical origin), SiHa (vaginal cells), and CaCo-2 (intestinal cells). In these experiments, the aforementioned cells are grown in 96-well plates, and at confluence, they were incubated with different concentrations of zinc tetra-ascorbo-camphorate compound referred to as C14 as disclosed herein in the range of 1 μM to 500 μM (n=4 repeats for each concentration). After treatment that was an incubation with C14 for a duration of exposure of 24 hours, the supernatant was removed and replaced by the solution XTT. KIT Tox-2 XTT Sigma Aldrich.

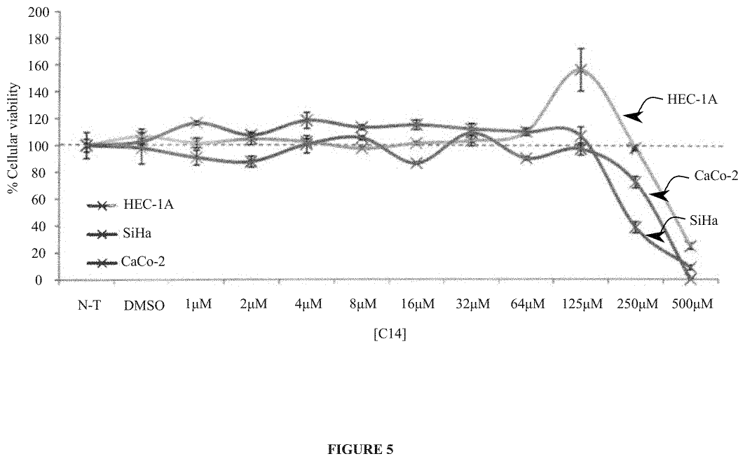

The results of the present experiment and example are illustrated in FIG. 5 demonstrating that C14 does not exhibit toxicity to epithelial cells at concentrations less than or equal to 125 μM.

### Example 5

### Impact of C14 on the Sealing of Epithelial Mucosa

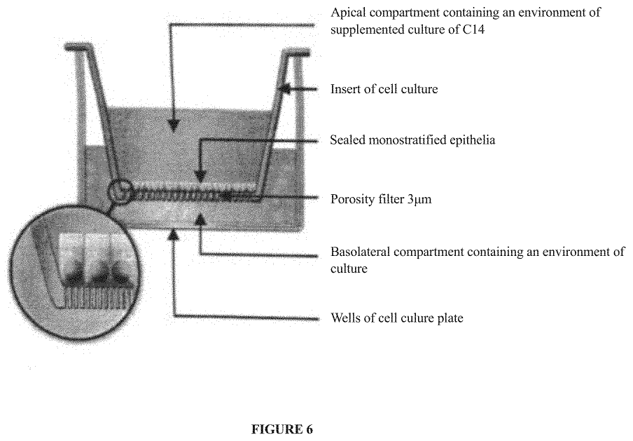

In this experiment, the measurement of the transepithelial electrical resistance (TEER) that allowed to evaluate the tightness of monostratified epithelial mucosa whereas a rupture of this resistance can lead to an increase in the transmission of the HIV virus was used to assess the impact of zinc tetra-ascorbo-camphorate compound referred to as C14 as disclosed herein on the sealing of epithelial mucosa. To assess the TEER, a cell culture system model based on the use of cell culture inserts as shown in FIG. 6 was used.

In this model, the cell culture inserts that had a porosity filter of 3 μm as shown and indicated in the illustration of FIG. 6 thereby allowed to mimic the apical (lumen) and basal (lamina propria) compartments, where the cell lines tested include HEC-1A and CaCo-2 as representatives for sealed monostratified epithelia. The cell culture inserts were in turn contained inside wells of a cell culture plate with basolateral compartment containing an environment of culture, and inside the cell culture insert, the apical compartment contains an environment of supplemented culture where, when the aforesaid cells were at confluence, the apical medium was replaced by a C14 solution of given concentration that were in a range from 1 μM to 500 μM (n=2 for each concentration), where the exposure time was of 24 hours.

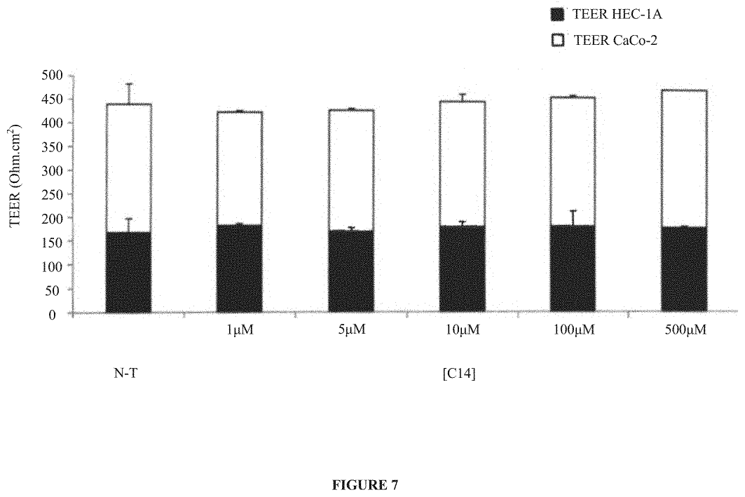

FIG. 7 provides the results of the aforementioned experiments showing in terms of TEER so measures that C14 as disclosed in the present invention does not affect the TEER of monostratified epithelia, even at high concentrations (500 μM).

### Example 6

### Dosage of Cytokines Related to Inflammation

In the experiment demonstrated in this example, the effect of zinc tetra-ascorbo-camphorate compound referred to as C14 as disclosed herein on the secretion of cytokines/chemokines as measures of inflammatory or anti-inflammatory effect was assessed. In the model as provided in this example, a method to assess the pro-inflammatory character of antivirals intended to be applied as mucosal topicals was used.

Epithelial cells are capable of secreting a set of molecules called cytokines and chemokines, which are responsible for the recruitment and activation of immune cells at the site of secretion. This process can be deleterious in the context of sexual transmission of HIV-1 because the recruited cells are the preferred targets of the virus. It has also been shown that pro-inflammatory conditions lead to an increase in the transcytosis mechanism of viral particles through the epithelium (Carreno & al, Cytokine, 2009). A microbicide candidate should therefore not promote inflammation by activating the production of this type of molecule by the cells directly in contact with the substance (epithelial cells). These secretions can be deleterious in the context of sexual transmission of HIV-1 or HPV and other papillomaviruses because the recruited cells are the preferred targets of the virus. A microbicide candidate must thus not promote inflammation by activating the production of these types of molecules by the cells in contact with the substance (epithelial cells). Therefore, in this example and experiment, the influence of C14 on inflammation in terms of assaying a large panel of 48 cytokines/chemokines (refer to Table 2 hereinbelow) related to inflammation as secreted by HEC-1A cells by MultiPlex assay was measured and the results were assessed to check whether there was any pro-inflammatory, no change, or anti-inflammatory effect by an exposure to C14 at different concentrations.

The cell culture model tested in this example, i.e., HEC-1A cells were grown on permeable media similar to the system as shown in FIG. 6. At confluence, the apical medium was replaced by a C14 solution of given concentration in the range from 1 μM to 500 μM (n=2 for each concentration), where the exposure time was of 24 hours. After 24 hours of incubation, a panel of 48 inflammation-related cytokines/chemokines is assayed by MultiPlex (BioPlex 200 BioRad) in the basal medium.

Table 2 hereinbelow show the 48 cytokines/chemokines included in the cytokine/chemokine multiplex assay as shown hereinbelow.

| | | | | | | | | | | | |
|---|---|---|---|---|---|---|---|---|---|---|---|
| IL-1a | IL-1b | IL-1ra | IL-2 | IL-2ra | IL-3 | IL-4 | IL-5 | IL-6 | IL-7 | IL-8 | IL-9 |
| IL-10 | IL-12p40 | IL-12p70 | IL-13 | IL-15 | IL-16 | IL-17 | IL-18 | TNF-α | TNF-β | IFN-α2 | IFN-γ |
| SDF-1α | G-CSF | GM-CSF | CTACK | GROα | FGF | HGF | TRAIL | Eotaxin | MCP-1 | MCP-3 | LIF |
| MIP-1α | MIP-1β | M-CSF | MIF | MIG | β-NGF | SCF | SCGF-β | PDGF-β | RANTES | VEGF | IP-10 |

The following Table 3 (parts a, b, c, and d) provides a summary of the assay results by aforementioned cytokines/chemokines multiplex/BioPlex assay as provides in the Table 2 above comparing the 24 hours of incubation and treatment with C14 solution of given concentration in the range from 1 μM to 500 μM (n=2 for each concentration) as compared with no treatment (N-T) as shown below, where the data represents the average of duplicates, and are expressed in pg/ml:

TABLE 3 (a)

| Cytokine- | | | | | | | | | | | | | | |
|---|---|---|---|---|---|---|---|---|---|---|---|---|---|---|
| chemokine | | | IL-1β | IL-1rα | IL-2 | IL-4 | IL-5 | IL-6 | IL-7 | IL-8 | IL-9 | IL-10 | IL-12 | IL-13 |
| N-T | | | 0.395 | 31.565 | 0.365 | 0.15 | 0 | 0.575 | 5.075 | 1883.86 | 34.295 | 4.105 | 123.27 | 4.95 |
| C14 | 500 | μM | 0.415 | 14.09 | 0.28 | 0.295 | 0 | 0.79 | 4.66 | 1219.13 | 30.4 | 4.165 | 121.285 | 4.555 |
| | 100 | μM | 0.34 | 10.3 | 0.02 | 0.33 | 0 | 0.255 | 4.495 | 783.255 | 22.305 | 3.505 | 99.355 | 3.735 |
| | 10 | μM | 0.33 | 14.085 | 0.305 | 0.235 | 0 | 0.575 | 3.665 | 1115.205 | 26.025 | 3.17 | 103.16 | 4.145 |
| | 5 | μM | 0.31 | 10.215 | 0.105 | 0.05 | 0 | 0.34 | 3.38 | 941.46 | 25.535 | 3.395 | 98.855 | 4.8 |
| | 0 | μM | 0.335 | 18.105 | 0.25 | 0.16 | 0 | 0.435 | 3.95 | 971.01 | 25.865 | 3.085 | 98.495 | 3.34 |

TABLE 3 (b)

| Cytokine- | | | | | | | | | | | | | | |
|---|---|---|---|---|---|---|---|---|---|---|---|---|---|---|
| chemokine | | | IL-15 | IL-17 | Eotaxin | FGF | G-CSF | GM-CSF | IFN-γ | IP-10 | MCP-1 | MIP-1α | PDGF-β | MIP-1β |
| N-T | | | 0 | 0 | 0 | 1.49 | 2.72 | 5.12 | 118.695 | 10.765 | 13.73 | 1.77 | 1.94 | 1.425 |
| C14 | 500 | μM | 0 | 0 | 3.21 | 1.925 | 2.3 | 7.53 | 98.775 | 10.765 | 5.165 | 2.04 | 6.19 | 1.655 |
| | 100 | μM | 0 | 0 | 0 | 1.635 | 1.885 | 9.51 | 89.85 | 0 | 5.3 | 1.77 | 3.58 | 1.26 |
| | 10 | μM | 0 | 0 | 0 | 1.53 | 1.99 | 6.65 | 76.375 | 7.165 | 7.55 | 2.065 | 2.805 | 1.555 |
| | 5 | μM | 0 | 0 | 0 | 1.415 | 1.99 | 6.24 | 95.43 | 4.855 | 5.105 | 2.105 | 0.865 | 1.09 |
| | 0 | μM | 0 | 0 | 0.87 | 1.515 | 1.885 | 7.005 | 94.255 | 6.19 | 7.26 | 2.25 | 1.235 | 1.32 |

TABLE 3 (c)

| Cytokine- | | | | | | | | | | | | | | |
|---|---|---|---|---|---|---|---|---|---|---|---|---|---|---|
| chemokine | | | RANTES | TNF-α | VEGF | IL-1α | IL-2rα | IL-3 | IL-12ρ40 | IL-16 | IL-18 | CTACK | GROα | HGF |
| N-T | | | 14.58 | 9.655 | 2026.52 | 0 | 4.97 | 6.785 | 0 | 0 | 0 | 4.29 | 5.5 | 6.22 |
| C14 | 500 | μM | 13.835 | 10.49 | 1814.38 | 0 | 4.39 | 6.785 | 0 | 0 | 0 | 3.425 | 2.75 | 6.22 |
| | 100 | μM | 12.205 | 5.465 | 1469.495 | 0 | 3.245 | 5.495 | 0 | 0 | 0 | 2.585 | 0 | 4.58 |
| | 10 | μM | 12.995 | 6.985 | 1530.905 | 0 | 3.825 | 6.14 | 0 | 0 | 0 | 3.45 | 0 | 5.43 |
| | 5 | μM | 12.27 | 5.5 | 1240.71 | 0 | 4.29 | 6.785 | 0 | 0 | 0 | 4.29 | 0 | 5.43 |
| | 0 | μM | 12.86 | 4.9 | 1384.84 | 0 | 4.06 | 6.785 | 0 | 0 | 0 | 4.29 | 2.75 | 3.63 |

TABLE 3 (d)

| Cytokine- | | | | | | | | | | | | | | |
|---|---|---|---|---|---|---|---|---|---|---|---|---|---|---|
| chemokine | | | IFN-α2 | LIF | MCP-3 | M-CSF | MIF | MIG | β-NGF | SCF | SCGF-β | SDF-1α | TNF-β | TRAIL |
| N-T | | | 4.08 | 0 | 0 | 4.21 | 10.29 | 3.09 | 0.905 | 0 | 144.975 | 5.705 | 0 | 2.65 |
| C14 | 500 | μM | 3.58 | 0 | 0 | 3.995 | 5.26 | 3.09 | 0.995 | 0 | 128.865 | 11.41 | 0 | 3.43 |
| | 100 | μM | 4.08 | 0 | 0 | 4.725 | 1.06 | 2.75 | 0.95 | 0 | 228.825 | 11.41 | 0 | 5.025 |
| | 10 | μM | 4.08 | 0 | 0 | 4.805 | 1.06 | 3.76 | 0.86 | 0 | 255.82 | 11.41 | 0 | 5.025 |
| | 5 | μM | 3.58 | 0 | 0 | 4.7 | 2.735 | 3.76 | 0.95 | 0 | 258.645 | 13.03 | 0 | 4.21 |
| | 0 | μM | 4.08 | 0 | 0 | 3.725 | 6.92 | 3.09 | 1.04 | 0 | 242.36 | 13.03 | 0 | 3.43 |

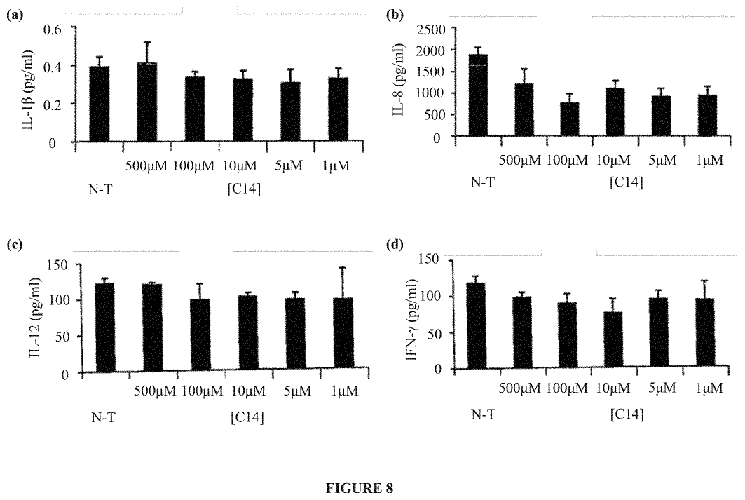

The aforementioned results in Table 3 demonstrates that C14 did not affect the production of any of the 48 cytokines/chemokines as listed in Table 2 and tested and assayed in this example of the present invention. In addition, FIG. 8 provides an illustrative representation of the data for selected cytokines/chemokines namely, IL-10, IL-8, IL-12, and IFN-γ using a cytokine-chemokine multiplex assay as enlisted in Table 2 (assays for 48 inflammation-related cytokines/chemokines by Multiplex assay (BioPlex 200 BioRad)) analysis when the cell line model, HEC-1A is incubated at confluence with zinc tetra-ascorbo-camphorate referred to as C14 at a concentration in a range from 1 μM to 500 μM (n=2 for each concentration). The results demonstrate that there is no increase in secretion by HEC-1A cells of the cytokine-chemokines assayed.

The results as demonstrated in this example demonstrate that zinc tetra-ascorbo-camphorate compound referred to as C14 as disclosed herein does not provoke an inflammatory response whatever the concentration. The C14 molecule is in favor of the modulation of the inflammatory response or even has anti-inflammatory properties in the mucosal context. The present example shows that C14 does not increase secretion by HEC-1A cells of the cytokine-chemokines assayed. The data and assessment as provided in this disclosure is in line with the literature about terpenoids.

It will be apparent to those skilled in the art that various modifications and variations can be made in the practice of the present invention without departing from the scope or spirit of the invention. Other embodiments of the invention will be apparent to those skilled in the art from considering of the specification and practice of the invention. It is intended that the specification and examples be considered as exemplary only, with a true scope and spirit of the invention being indicated by the following claims.

What is claimed is:

1. A method for preventive and/or curative treatment of viral infections caused by viruses of the family Papillomaviridae in a subject, said method comprising the step of administering a therapeutically effective amount of a composition to the subject, wherein the composition comprises at least one metal ascorbo-camphorate compound, a physiologically acceptable aqueous solvent, and at least one physiologically acceptable excipient, and wherein the metal ascorbo-camphorate compound is Zinc tetra-ascorbo-camphorate referred to as C14.

2. The method of claim 1, wherein the subject is a human.

3. The method of claim 1, wherein the viruses are human papilloma viruses (HPV) including oncogenic papillomaviruses consisting of the HPV-16 genotype, the HPV-18 genotype, or a combination thereof.

4. The method of claim 1, wherein the physiologically acceptable aqueous solvent is selected from a group of solvents consisting of water, a mixture of water and an alcohol, a mixture of water and another polar solvent, or a combination thereof, and wherein the alcohol or the polar solvent is physiologically acceptable.

5. The method of claim 1, wherein the at least one physiologically acceptable excipient is an excipient for packaging in a galenic form of a gel or an ointment type for administering the therapeutically effective amount of a composition to the subject via the skin, and/or mucous membranes.

6. The method of claim 1, wherein the at least one physiologically acceptable excipient is an excipient for packaging in a solid dosage of tablets, capsules or sachets in powder form for administering the therapeutically effective amount of the composition to the subject via dissolution in water for external application and/or for ingestion orally.

7. The method of claim 1, wherein the at least one physiologically acceptable excipient is an excipient for packaging in a galenic form of an ovule or a capsule or a vaginal ring or a vaginal bulb and/or pump or an embedded sanitary pad type for administering the therapeutically effective amount of a composition to the subject via the vaginal route.

8. The method of claim 1, wherein the therapeutically effective amount of the composition comprising the Zinc tetra-ascorbo-camphorate referred to as C14 is administered to the subject in a liquid form with the Zinc tetra-ascorbo-camphorate referred to as C14 dissolved in the physiologically acceptable aqueous solvent in the administered composition is at a dilution in a range between 1:500,000 (a five hundred thousands) and 1:10 (a tenth), and wherein the physiologically acceptable aqueous solvent is water.

9. The method of claim 1, wherein the therapeutically effective amount of the composition comprising the Zinc tetra-ascorbo-camphorate referred to as C14 is administered to the subject in a solid form with the Zinc tetra-ascorbo-camphorate referred to as C14 at a dosage in a range between 20 mg and 3 g per day for a human subject, and wherein the said dosage is given as a single dose or as multiple divided doses for a cumulative dosage of a range between 20 mg and 3 g per day for the human subject.

10. The method of claim 1, wherein the therapeutically effective amount of the composition is administered to the subject by a route of administration selected from a group consisting of intravenous route, intraperitoneal route, oral route, intranasal route, subcutaneous route, intrathecal route, intraventricular route, intramuscular route, by instillation route via a vaginal bulb and/or pump, by perfusion route, and by vaginal ring route, and wherein the at least one physiologically acceptable excipient comprised in said composition provides the packaging for administration via the selected route of administration.

11. A pharmaceutical composition directed against viral infections caused by viruses of the family Papillomaviridae, the pharmaceutical composition comprising:
(a) a metal ascorbo-camphorate compound;
(b) a physiologically acceptable aqueous solvent; and
(c) at least one physiologically acceptable excipient,
wherein the metal ascorbo-camphorate compound is a metal tetra-ascorbo-camphorate,
wherein the metal ascorbo-camphorate compound comprises a metal ion selected from the group comprising metals of the series 2 of the Mendeleev classification including Zinc, Copper, Gold, Silver, and Magnesium,
wherein the metal ascorbo-camphorate compound is soluble in the physiologically acceptable aqueous solvent,
wherein the viruses of the family Papillomaviridae are human papilloma viruses (HPV) including oncogenic papillomaviruses consisting of the HPV-16 genotype, the HPV-18 genotype, or a combination thereof, and
wherein the at least one physiologically acceptable excipient comprised in said composition provides the packaging for administration via a route of administration in a subject for a route of administration selected from a group consisting of intravenous route, intraperitoneal route, oral route, intranasal route, subcutaneous route, intrathecal route, intraventricular route, intramuscular route, by instillation route via a vaginal bulb and/or pump, by perfusion route, and by vaginal ring route.

12. The pharmaceutical composition of claim 11, wherein the metal ascorbo-camphorate compound is Zinc tetra-ascorbo-camphorate referred to as C14.

13. The pharmaceutical composition of claim 11, wherein the physiologically acceptable aqueous solvent is selected from a group of solvents consisting of water, a mixture of water and an alcohol, a mixture of water and another polar solvent, or a combination thereof, and wherein the alcohol or the polar solvent is physiologically acceptable.

14. The pharmaceutical composition of claim 11, wherein the at least one physiologically acceptable excipient is selected from a group of excipients consisting of an excipient for packaging in a galenic form of a gel or an ointment type for administering the therapeutically effective amount of a composition to the subject via the skin, and/or mucous membranes, or an excipient for packaging in a solid dosage of tablets, capsules or sachets in powder form for administering the therapeutically effective amount of the composition to the subject via dissolution in water for external application and/or for ingestion orally, or an excipient for packaging in a galenic form of an ovule or a capsule or a vaginal ring or a vaginal bulb and/or pump or an imbibed sanitary pad type for administering the therapeutically effective amount of a composition to the subject via the vaginal route.

15. A method for preparation of a pharmaceutical composition for administering a therapeutically effective amount of said composition to a subject directed against viral infections caused by viruses of the family Papillomaviridae, said method comprising the steps of:
preparing camphoric acid by oxidation of camphor, wherein the camphor is obtained from the alpha-pinene contained in turpentine which is in turn obtained from the resinous juice that flows from maritime pine;
preparing zinc camphorate from the prepared camphoric acid in the steps of:
solubilizing 200 mg of the prepared camphoric acid in 1 g of ethanol at 90° to obtain a first mixture, adding 0.5 g of ultrapure water to the first mixture to obtain a second mixture, mixing 81.3 mg of zinc oxide with 1 g of ultrapure water by stirring to form a frying solution, pouring in the second mixture comprising camphoric acid slowly into said frying solution comprising zinc oxide while stirring intermittently in phases of stirring to obtain a third mixture, wherein in between each of the phases of stirring, heating the third mixture slightly in an oven, obtaining a precipitate of zinc camphorate and recovering it by extraction of the solvent either by heating or by freeze-drying;
preparing zinc tetra-ascorbate compound from the prepared zinc camphorate by mixing the prepared zinc camphorate in a powder form to an aqueous solution of ascorbic acid to obtain a fourth mixture, wherein the molar ratio of ascorbic acid/zinc camphorate equal to 4; wherein the mixing is carried out in a dilute medium corresponding to an amount of water in a range of between 20 and 40 equivalents mL/mmol with respect to the zinc camphorate, and wherein the fourth mixture is kept under moderate stirring and under an inert atmosphere and protected from light to avoid oxidation of the ascorbic acid, until the zinc camphorate is completely dissolved to obtain zinc tetra-ascorbo-camphorate, wherein the mixing is carried out at a temperature below 25° C., wherein the inert atmosphere is maintained under argon gas, and wherein the moderate stirring is carried out for a total of 3 hours to completely dissolve zinc camphorate which reacts with the dissolved ascorbic acid to obtain a zinc tetra-ascorbo-camphorate in solution;
recovering said zinc tetra-ascorbo-camphorate compound in solution into a powder form by cold freeze-drying in order to extract the solvent under vacuum and cold and the obtained zinc tetra-ascorbo-camphorate compound referred to as C14 is lyophilized and kept in a dark place to obtain lyophilized zinc tetra-ascorbo-camphorate compound referred to as C14; and
mixing the lyophilized zinc tetra-ascorbo-camphorate compound referred to as C14 with a physiologically acceptable aqueous solvent to dissolve it, and at least one physiologically acceptable excipient to obtain the pharmaceutical composition for administering a therapeutically effective amount of said composition to a subject directed against viral infections caused by viruses of the family Papillomaviridae.

16. The method of claim 15, wherein the physiologically acceptable aqueous solvent is selected from a group of solvents consisting of water, a mixture of water and an alcohol, a mixture of water and another polar solvent, or a combination thereof, and wherein the alcohol or the polar solvent is physiologically acceptable.

17. The method of claim 15, wherein the at least one physiologically acceptable excipient is selected from a group of excipients consisting of an excipient for packaging in a galenic form of a gel or an ointment type for administering the therapeutically effective amount of a composition to the subject via the skin, and/or mucous membranes, or an excipient for packaging in a solid dosage of tablets, capsules or sachets in powder form for administering the therapeutically effective amount of the composition to the subject via dissolution in water for external application and/or for ingestion orally, or an excipient for packaging in a galenic form of an ovule or a capsule or a vaginal ring or a vaginal bulb and/or pump or an imbibed sanitary pad type for administering the therapeutically effective amount of a composition to the subject via the vaginal route.

18. The method of claim 15, wherein the viruses of the family Papillomaviridae are human papilloma viruses (HPV) including oncogenic papillomaviruses consisting of the HPV-16 genotype, the HPV-18 genotype, or a combination thereof

19. The pharmaceutical composition of claim 11 for use in the preventive and/or curative treatment of viral infections caused by viruses of the family Papillomaviridae in a subject, the pharmaceutical composition comprising:
(a) a metal ascorbo-camphorate compound;
(b) a physiologically acceptable aqueous solvent; and
(c) at least one physiologically acceptable excipient,
wherein the metal ascorbo-camphorate compound is Zinc tetra-ascorbo-camphorate referred to as C14,
wherein the metal ascorbo-camphorate compound is soluble in a physiologically acceptable aqueous solvent,
wherein the physiologically acceptable aqueous solvent is selected from a group of solvents consisting of water, a mixture of water and an alcohol, a mixture of water and another polar solvent, or a combination thereof, and wherein the alcohol or the polar solvent is physiologically acceptable,
wherein the viruses of the family Papillomaviridae are human papilloma viruses (HPV) including oncogenic papillomaviruses consisting of the HPV-16 genotype, the HPV-18 genotype, or a combination thereof, and
wherein the pharmaceutical composition is administered to the subject by a route of administration selected from a group consisting of intravenous route, intraperitoneal route, oral route, intranasal route, subcutaneous route, intrathecal route, intraventricular route, intramuscular route, by instillation route via a vaginal bulb and/or pump, by perfusion route, and by vaginal ring route, and wherein the at least one physiologically acceptable excipient comprised in said composition provides the packaging for administration via the route of administration.

20. The pharmaceutical composition of claim 19, wherein the at least one physiologically acceptable excipient is selected from a group of excipients consisting of an excipient for packaging in a galenic form of a gel or an ointment type for administering the therapeutically effective amount of a composition to the subject via the skin, and/or mucous membranes, or an excipient for packaging in a solid dosage of tablets, capsules or sachets in powder form for administering the therapeutically effective amount of the composition to the subject via dissolution in water for external application and/or for ingestion orally, or an excipient for packaging in a galenic form of an ovule or a capsule or a vaginal ring or a vaginal bulb and/or pump or an imbibed sanitary pad type for administering the therapeutically effective amount of a composition to the subject via the vaginal route.
